(19) United States
(12) Patent Application Publication
(10) Pub. No.: US 2020/0055897 A1
(43) Pub. Date: Feb. 20, 2020

(54) MODIFIED PEPTOIDS

(71) Applicant: Technion Research & Development Foundation Limited, Haifa (IL)

(72) Inventors: Galia MAAYAN, Haifa (IL); Kaniraj Jeya PRATHAP, Kanyakumari District (IN)

(73) Assignee: Technion Research & Development Foundation Limited, Haifa (IL)

(21) Appl. No.: 16/607,733

(22) PCT Filed: Apr. 26, 2018
(86) PCT No.: PCT/IL2018/050464

§ 371 Date: Oct. 24, 2019

(30) Foreign Application Priority Data
Apr. 26, 2017 (IL) 251942

(51) Int. Cl. C07K 7/06; A61K 9/08

(52) U.S. Cl. CPC C07K 7/06; A61K 9/08

(57) ABSTRACT

Peptoids are described herein, as well as compositions comprising an aqueous liquid and a peptoid soluble in the aqueous liquid. The peptoids comprise a plurality of N-substituted glycine residues and/or N-substituted β-alanine residues, and at least one heteroalicyclic residue having a general formula:

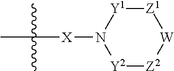

wherein W, X, Y¹, Y², Z¹ and Z² are as defined herein.

### FIELD AND BACKGROUND OF THE INVENTION

The present invention, in some embodiments thereof, relates to peptidomimetics, and more particularly, but not exclusively, to peptoids having enhanced water solubility.

N-substituted glycine oligomers are commonly referred to as “peptoids”, and may serve as peptide mimics which are both biocompatible and resistant to proteolytic degradation, as well as being relatively easy to synthesize [Zuckermann et al., J. Am. Chem. Soc. 1992, 114:10646-10647]. Some peptoid sequences with chiral centers adjacent to main chain nitrogen atoms have an ability to fold into stable and well-defined secondary structures resembling peptide alpha helices [Kirshenbaum et al., Proc. Natl. Acad. Sci. 1998, 95:4303-4308]. Hydrogen bonding by N-aryl side chains with the peptoid backbone has been reported to play an important role in peptoid folding [Stringer et al., J. Org. Chem. 2010, 75:6068-6078]. Peptoids have been employed to bind to therapeutically relevant proteins and to metal ions [Lee et al., J. Am. Chem. Soc. 2008, 130:8847-8855; Knight et al., J. Am. Chem. Soc. 2013, 135:17488-17493] and in enantioselective catalysis [Maayan et al., Proc. Natl. Acad. Sci. USA 2009, 106:13679-13684]. In addition, in comparison to peptides, peptoids exhibit greater cell permeability, proteolytic resistance, and tolerance towards high salt concentration, organic solvents, and various pH conditions [Kwon & Kodadek, Am. Chem. Soc. 2007, 129:1508-1509; Miller et al., Drug Development Research 1995, 35:20-32].

However, the utilization of peptoids for biological and medicinal applications is still limited, as many sequences that hold potential as drugs consist of hydrophobic monomers which result in peptoids that are not soluble in water. Currently, water-soluble peptoids are constructed via the incorporation of polar hydrophilic pendant groups within the peptoid sequence. This approach, however, requires that at least 66% of the pendant monomers will be hydrophilic and these are typically spread evenly along the sequence to ensure water solubility of the peptoid [Lee et al., J. Am. Chem. Soc. 2008, 130:8847-8855; Knight et al., J. Am. Chem. Soc. 2013, 135:17488-17493].

Suwal & Kodadek describe incorporation of a piperazine or 2-methylpiperazine monomer in peptoids in order to enhance the diversity of a peptoid library [Org. Biomol. Chem. 2014, 12:5831-5834], as well as techniques for incorporating oxopiperazine [Org. Biomol. Chem. 2013, 11:2088-2092] or diketopiperazine [Org. Biomol. Chem. 2014, 12:5831-5834] units in peptoids.

Additional background art includes Barron & Zuckermann [Curr. Opin. Chem. Biol. 1999, 3:681-687]; Baskin et al. [Chem. Commun. 2016, 52:10350-10353]; Baskin & Maayan [Biopolymers (Peps. Sci.) 2015, 104:577-584]; Baskin & Maayan [Chem. Sci. 2016, 7:2809-2820]; Brown et al. [Biochemistry 2008, 47:1808-1818]; Burkoth et al. [J. Am. Chem. Soc. 2003, 125:8841-8845]; Chandra Mohan et al. [J. Catal. 2017, 355:139-144]; Chongsiriwatana et al. [Proc. Natl. Acad. Sci. USA 2008, 105:2794-2799]; Caumes et al. [J. Am. Chem. Soc. 2012, 134:9553-9556]; Crapster et al. [Angew. Chem. Int. Ed. 2013, 52:5079-5084]; Czyzewski et al. [PLoS ONE 2016, 11:e013596]; Darapaneni et al. [Org. Biomol. Chem. 2018, 16:1480-1488]; Della Sala et al. [Org. Biomol. Chem. 2013, 11:726-731]; Doran et al. [Bioorg. Med. Chem. Lett. 2015, 25:4910-4917]; Fuller et al. [Peps. Sci. 2011, 96:627-638]; Fuller et al. [Org. Lett. 2013, 15:5118-5121]; Gorske et al. [J. Am. Chem. Soc. 2007, 129:8928-8929]; Gorske et al. [J. Am. Chem. Soc. 2009, 131:16555-16567]; Hara et al. [J. Am. Chem. Soc. 2006, 128:1995-2004]; Huang et al. [Proc. Natl. Acad. Sci. USA 2012, 109:19922-19927]; Huang et al. [PLoS ONE 2014, 9:e90397]; Kaniraj & Maayan [Org. Lett. 2015, 17:2110-2113]; Knight et al. [Chem. Sci. 2015, 6:4042-4048]; Laursen et al. [Nat. Commun. 2015, 6:7013]; Laursen et al. [Acc. Chem. Res. 2015, 48:2696-2704]; Maayan et al. [Chem. Commun. 2009, 1:56-58]; Maayan & Liu [Biopolymers (Peps. Sci.) 2011, 96:679-687]; Mojsoska et al. [Antimicrob. Agents Chemother. 2015, 59:4112-4120]; Nguyen et al. [Science 1998, 282, 2088-2092]; Norgren et al. [Org. Lett. 2006, 8:4533-4536]; Olsen et al. [Amino Acids 2008, 34:465-471]; Patch & Barron [J. Am. Chem. Soc. 2003, 125:12092-12093]; Paul et al. [Org. Lett. 2012, 14:926-929]; Paul et al. [J. Am. Chem. Soc. 2011, 133:10910-10919]; Prathap & Maayan [Chem. Commun. 2015, 51:11096-11099]; Roy et al. [Org. Lett. 2013, 15:2246-2249]; Roy et al. [J. Am. Chem. Soc. 2017, 139:13533-13540]; Sanborn et al. [Biopolymers 2002, 63:12-20]; Sarma & Kodadek [ACS Comb. Sci. 2012, 14:558-564]; Sarma et al. [Chem. Commun. 2011, 47:10590-10592]; Schettini et al. [Eur. J. Org. Chem. 2014, 2014:7793-7797]; Schettini et al. [J. Org. Chem. 2016, 81:2494-2505]; Shah et al. [J. Am. Chem. Soc. 2008, 130:16622-16632]; Shin & Kirshenbaum [Org. Lett. 2007, 9:5003-5006]; Statz et al. [J. Am. Chem. Soc. 2005, 127:7972-7973]; Stringer et al. [J. Am. Chem. Soc. 2011, 133:15559-15567]; Tigger-Zaborov & Maayan [J. Colloid Interface Sci. 2017, 508:56-64]; Udugamasooriya et al. [J. Am. Chem. Soc. 2008, 130:5744-5752]; Vanderstichele & Kodadek [Alzheimer's Res. Ther. 2014, 6:23]; Wender et al. [Proc. Natl. Acad. Sci. USA 2000, 97:13003-13008]; Wu et al. [J. Am. Chem. Soc. 2001, 123:2958-2963]; Wu et al. [J. Am. Chem. Soc. 2001, 123:6778-5784]; Wu et al. [J. Am. Chem. Soc. 2003, 125:13525-13530]; Wu & Kirshenbaum [J. Am. Chem. Soc. 2015, 137:6312-6319]; Zabrodski et al. [Synlett 2015, 26:461-466]; Zborovsky et al. [Chemistry 2018, 24:1159-1167]; and Seo et al. [in: Peptoids-Synthesis, Characterization, and Nanostructures. Ducheyne, Healy, Hutmacher, Grainger, Kirkpatrick (eds.) Comprehensive Biomaterials, 2011, vol. 2, pp. 53-76, Elsevier].

### SUMMARY OF THE INVENTION

According to an aspect of some embodiments of the invention, there is provided a peptoid comprising a plurality of N-substituted glycine residues and/or N-substituted β-alanine residues, and at least one heteroalicyclic residue having a general formula:

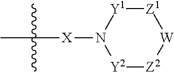

wherein:

W is selected from the group consisting of NR₁₅, O, S, S(═O), S(═O)₂, and a nitrogen atom attached by a covalent (e.g., amide) bond to another portion of the peptoid;

X is selected from the group consisting of —C(═O)—CR₁R₂— and —NR_{16—}, or X is absent;

Y¹ is CR₃R₄ or C═O;

Y² is CR₅R₆ or C═O;

Z¹ is CR₇R₈ or C═O;

Z² is CR₉R₁₀ or CR₁₁R₁₂—CR₁₃R₁₄;

R₁-R₁₄ are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, phosphate, phosphonyl, phosphinyl, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, hydrazine, and amino; and

R₁₅ and R₁₆ are each independently selected from the group consisting of hydrogen, alkyl, alkenyl, alkynyl, cycloalkyl, aryl, heteroaryl, heteroalicyclic, carbonyl, thiocarbonyl, C-amido, and C-carboxy,

or alternatively, any two of R₁-R₁₆ together form a 5-, 6- or 7-membered alicyclic or heteroalicyclic ring.

According to an aspect of some embodiments of the invention, there is provided a peptoid comprising at least three N-substituted glycine residues and/or N-substituted β-alanine residues, and at least one heteroalicyclic residue having a general formula:

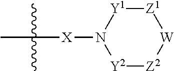

wherein: W is selected from the group consisting of NR₁₅, O, S, S(═O), S(═O)₂, and a nitrogen atom attached by a covalent bond to another portion of the peptoid;

X is selected from the group consisting of —C(═O)—CR₁R₂— and —NR_{16—}, or X is absent;

Y¹ is CR₃R₄;

Y² is CR₅R₆;

Z¹ is CR₇R₈;

Z² is CR₉R₁₀ or CR₁₁R₁₂—CR₁₃R₁₄;

R₁-R₁₄ are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, phosphate, phosphonyl, phosphinyl, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, hydrazine, and amino; and

R₁₅ and R₁₆ are each independently selected from the group consisting of hydrogen, alkyl, alkenyl, alkynyl, cycloalkyl, aryl, heteroaryl, heteroalicyclic, carbonyl, thiocarbonyl, C-amido, and C-carboxy,

or alternatively, any two of R₁-R₁₆ together form a 5-, 6- or 7-membered alicyclic or heteroalicyclic ring,

wherein at least one of the at least one heteroalicyclic residue is directly attached via an amide bond to a backbone nitrogen atom or a backbone carbonyl of at least one of the N-substituted glycine residues and/or N-substituted β-alanine residues.

According to an aspect of some embodiments of the invention, there is provided a composition comprising a peptoid described herein and an aqueous liquid, the peptoid being soluble in the aqueous liquid.

According to some embodiments of any of the embodiments of the invention, R₁-R₁₄ are each independently selected from the group consisting of hydrogen, C₁₋₄-alkyl, C₂₋₄-alkenyl, C₂₋₄-alkynyl, phenyl, C₁₋₄-alkoxy, C₁₋₄-thioalkoxy, C₁₋₄-carbonyl, C₁₋₄-thiocarbonyl, C₁₋₄—N-amido, C₁₋₄—O-carboxy, nitro, —S(═O)₂OH, —O—S(═O)₂OH, —S(═O)2NH₂, —C(═O)NH₂ and —C(═O)OH.

According to some embodiments of any of the embodiments of the invention, R₁-R₁₆ are each independently hydrogen or methyl.

According to some embodiments of any of the embodiments of the invention, at least one of the at least one heteroalicyclic residue is directly attached via an amide bond to a backbone nitrogen atom or a backbone carbonyl of at least one of the N-substituted glycine residues and/or N-substituted β-alanine residues.

According to some embodiments of any of the embodiments of the invention, at least one heteroalicyclic residue is a terminal residue.

According to some embodiments of any of the embodiments of the invention, the W of the terminal residue is NH, O, S, S(═O) or S(═O)₂, or the X of the terminal residue is a part of a HOC(═O)—CR₁R₂— or H₂NC(═O)—CR₁R₂— group.

According to some embodiments of any of the embodiments of the invention, W is NR₁₅ or the nitrogen atom attached by a covalent bond to another portion of the peptoid.

According to some embodiments of any of the embodiments of the invention, Z² is CR₁₁R₁₂—CR₁₃R_{14.}

According to some embodiments of any of the embodiments of the invention, R₃-R₈ and Ru₁₁-R₁₄ are each hydrogen.

According to some embodiments of any of the embodiments of the invention, Y¹ is CR₃R₄ and Y² is CR₅R_{6.}

According to some embodiments of any of the embodiments of the invention, Z¹ is CR₇R₈.

According to some embodiments of any of the embodiments of the invention, the peptoid comprises at least two of the heteroalicyclic residue.

According to some embodiments of any of the embodiments of the invention, the at least two of the heteroalicyclic residue are attached to one another.

According to some embodiments of any of the embodiments of the invention, the peptoid comprises at least one of the heteroalicyclic residue per 750 Da of the total weight of the peptoid.

According to some embodiments of any of the embodiments of the invention, the peptoid comprises no more than two of the heteroalicyclic residue per 750 Da of the total weight of the peptoid.

According to some embodiments of any of the embodiments of the invention, a ratio of a number of the heteroalicyclic residue in the peptoid to a total number of the N-substituted glycine residues and N-substituted β-alanine residues in the peptoid is no more than 1:1.

According to some embodiments of any of the embodiments of the invention, the peptoid comprises at least three of the N-substituted glycine residues and/or N-substituted β-alanine residues described herein.

According to some embodiments of any of the embodiments of the invention, R₃-R₁₄ are each independently selected from the group consisting of hydrogen, alkyl, aryl, and C-amido, or alternatively, R₃ and R₅, and/or R₇ and R₉, together form a 5-, 6- or 7-membered heteroalicyclic ring.

According to some embodiments of any of the embodiments of the invention, R₃-R₁₄ are each hydrogen.

According to some embodiments of any of the embodiments of the invention, R₁, R₂ and R₁₆ are each hydrogen.

According to some embodiments of any of the embodiments of the invention, the peptoid has a water-solubility of at least 200 mg/liter.

According to some embodiments of any of the embodiments of the invention, a water-solubility of a corresponding peptoid lacking the at least one heteroalicyclic residue is less than 100 mg/liter.

According to some embodiments of any of the embodiments of the invention, a water-solubility of the peptoid is at least 150% of a water-solubility of a corresponding peptoid lacking the at least one heteroalicyclic residue.

According to some embodiments of any of the embodiments of the invention, the peptoid is characterized by a helical structure in aqueous solution.

According to some embodiments of any of the embodiments of the invention, a corresponding peptoid lacking the at least one heteroalicyclic residue is characterized by a helical structure in acetonitrile.

According to some embodiments of any of the embodiments of the invention, at least a portion of the N-substituted glycine residues and/or N-substituted β-alanine residues comprise a side chain which comprises a moiety selected from the group consisting of aryl, heteroaryl, cycloalkyl and heteroalicyclic.

According to some embodiments of any of the embodiments of the invention, the side chain is an alkyl substituted at a position 1 or 2 thereof, relative to the attachment point of the alkyl to a nitrogen atom of the N-substituted glycine residues and/or the N-substituted β-alanine residues, by the moiety.

According to some embodiments of any of the embodiments of the invention, the alkyl is methyl or ethyl.

According to some embodiments of any of the embodiments of the invention, the side chain is chiral.

According to some embodiments of any of the embodiments of the invention, the plurality of N-substituted glycine residues and/or N-substituted β-alanine residues consists of N-substituted glycine residues.

According to some embodiments of any of the embodiments of the invention relating to a composition, a concentration of the peptoid in the aqueous liquid is at least 10 mg/liter.

According to some embodiments of any of the embodiments of the invention relating to a composition, a pH of the aqueous liquid solution is in a range of from 5 to 9.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents the structure of a piperazine-modified peptoid (P2) according to some embodiments of the invention, as well as the structures of peptoids modified by alternative polar groups (SP1-SP4).

FIG. 2 is a bar graph showing the water solubility of modified peptoids presented in FIG. 1.

FIG. 3 presents a scheme depicting synthesis of a peptoid monomer (PM) and peptoid dimer (PD) from a peptoid and piperazine, as well as an optimization table showing relative amounts of PM and PD under various conditions ([b] 10 mg resin, DMF, rt, 35 minutes, 50 μl water added; [c] 50 μl water added).

FIGS. 4A and 4B present a proton-NMR spectrum of the piperazine-containing compound PD′ (FIG. 4A) and a depiction of the 3-dimensional structure of PD′, as determined by single crystal X-ray analysis (FIG. 4B).

FIG. 5 presents the structure of a control peptoid P1, as well as of exemplary peptoids (P2-P4) obtained by modifying peptoid P1 by incorporating a piperazine group therein according to some embodiments of the invention.

FIG. 6 is a bar graph showing the water solubility of peptoids presented in FIG. 5;

FIG. 7 presents circular dichroism (CD) spectra of the exemplary peptoid P2, as well as and a bar graph (inset) showing water solubility of P2, as pH 4.5, 7.5 and 10.5.

FIG. 8 presents a scheme showing a structure of the exemplary piperazine-containing peptoids P2, P5 and P6.

FIG. 9 presents circular dichroism (CD) spectra of the exemplary peptoids P2, P5 and P6 at pH 10.5, as well as and a bar graph (inset) showing water solubility of P2, P5 and P6 at pH 10.5.

FIG. 10 presents a scheme showing intramolecular hydrogen bonding of a piperazine moiety (attached to a peptoid) in a boat conformation and of a homopiperazine moiety (attached to a peptoid) in a twist chair conformation.

FIG. 11 presents a scheme showing a structure of the exemplary homopiperazine-containing peptoids P7 and P8.

FIG. 12 presents the structure of hydrophobic peptoids P9, P10 and P11, which were later modified according to some embodiments of the invention.

FIGS. 13A and 13B present circular dichroism (CD) spectra (FIG. 13A) of the exemplary peptoid P21 at concentrations of 40, 60, 80, 100, 120, 140, 160 and 180 μM, as well as a plot (FIG. 13B) of the negative molar ellipticity at 228 nm in the spectra as a function of P21 concentration.

FIG. 14 presents fluorescence emission spectra of the exemplary peptoids P18 and P21 in water (at a concentration of 40 μM; fluorescence emission in arbitrary units).

FIGS. 15A and 15B present circular dichroism (CD) spectra (FIG. 15A) of the exemplary peptoid P2 at concentrations of 40, 60, 80, 100, 120, 140, 160 and 180 μM, as well as a plot (FIG. 15B) of the negative molar ellipticity at 228 nm in the spectra as a function of P2 concentration.

FIGS. 16A and 16B present circular dichroism (CD) spectra (FIG. 16A) of the exemplary peptoid P5 at concentrations of 40, 60, 80, 100, 120, 140, 160 and 180 μM, as well as a plot (FIG. 16B) of the negative molar ellipticity at 228 nm in the spectra as a function of P5 concentration.

FIGS. 17A and 17B present circular dichroism (CD) spectra (FIG. 17A) of the exemplary peptoid P8 at concentrations of 40, 60, 80, 100, 120, 140, 160 and 180 μM, as well as a plot (FIG. 17B) of the negative molar ellipticity at 228 nm in the spectra as a function of P8 concentration.

FIG. 18 presents circular dichroism (CD) spectra of the exemplary piperazine-containing water-soluble peptoids P2 and P3 in acetonitrile and in water, as well as the CD spectrum in acetonitrile of the corresponding water-insoluble peptoid P1 without piperazine (x-axis represents wavelength in nm units).

FIG. 19 presents circular dichroism (CD) spectra in water of the exemplary peptoid P2 at temperatures ranging from 0° C. to 90° C. (x-axis represents wavelength in nm units).

FIG. 20 presents circular dichroism (CD) spectra of the exemplary homopiperazine-containing water-soluble peptoids P7 and P8 in acetonitrile and in water, as well as the CD spectrum in acetonitrile of the corresponding water-insoluble peptoid P1 without piperazine (x-axis represents wavelength in nm units).

FIG. 21 presents circular dichroism (CD) spectra of the exemplary piperazine-containing water-soluble peptoids P18 and P21 in acetonitrile and in water, as well as the CD spectrum in acetonitrile of the corresponding water-insoluble peptoid P9 without piperazine (x-axis represents wavelength in nm units).

FIG. 22 presents circular dichroism (CD) spectra of the exemplary water-soluble β-peptoids P26 and P27 (containing two and three piperazine residues, respectively) in acetonitrile and in water, as well as the CD spectra in acetonitrile of the corresponding β-peptoids P24 (without piperazine) and P25 (with one piperazine residue).

FIG. 23 presents circular dichroism (CD) spectra of the exemplary water-soluble azapeptoids P29, P30 and P31 (containing one, two and three piperazine residues, respectively) in acetonitrile and in water, as well as the CD spectrum in acetonitrile of the corresponding water-insoluble azapeptoid P28 without piperazine.

FIG. 24 presents the structure of a water-insoluble copper-binding peptoid P32, as well as of exemplary water-soluble peptoids (P33 and P34) obtained by modifying peptoid P32 by incorporating a piperazine group therein according to some embodiments of the invention.

FIG. 25 presents circular dichroism (CD) spectra of the exemplary peptoid P34 in the form of a free peptoid and in the form of a Cu²⁺-complex thereof.

FIG. 26 presents UV-visible absorption spectra of the exemplary peptoid P33 in the form of a free peptoid, in the form of a Cu²⁺-complex thereof, and upon contact with an equimolar mixture of Cu²⁺, Co²⁺, Zn²⁺, Mn²⁺, Ni²⁺ and Fe³⁺, at a molar ratio of 1:10 (peptoid:Cu²⁺).

FIG. 27 presents UV-visible absorption spectra of the exemplary peptoid P34 in the form of a free peptoid, in the form of a Cu²⁺-complex thereof, and upon contact with an equimolar mixture of Cu²⁺, Co²⁺, Zn²⁺, Mn²⁺, Ni²⁺ and Fe³⁺, at a molar ratio of 1:10 (peptoid:Cu²⁺).

FIG. 28 presents the structures of exemplary piperazine-like compounds which may be incorporated into a modified peptoid according to some embodiments of the invention.

### DESCRIPTION OF SPECIFIC EMBODIMENTS OF THE INVENTION

The present invention, in some embodiments thereof, relates to peptidomimetics, and more particularly, but not exclusively, to peptoids having enhanced water solubility.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventor has uncovered that residues of piperazine and related heteroalicyclic compounds are surprisingly effective at increasing water solubility of hydrophobic peptoids, for example, as compared with other hydrophilic residues. The inventor has further uncovered that modification of hydrophobic peptoids with such residues is an effective technique for providing water solubility to such peptoids with little modification to the side chain sequence of the peptoids, thereby facilitating conservation of peptoid properties.

While reducing the present invention to practice, the inventor has uncovered that introducing piperazine moieties to various peptoids results in piperazine-containing water-soluble peptoids that maintain a helical structure and functionality of corresponding non-modified peptoids, and can even enhance stability of the peptoid helical structure.

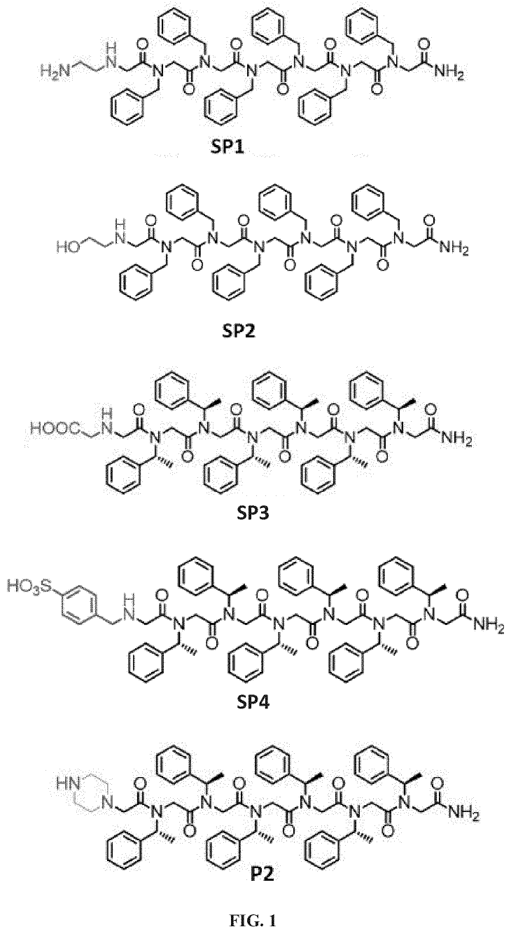

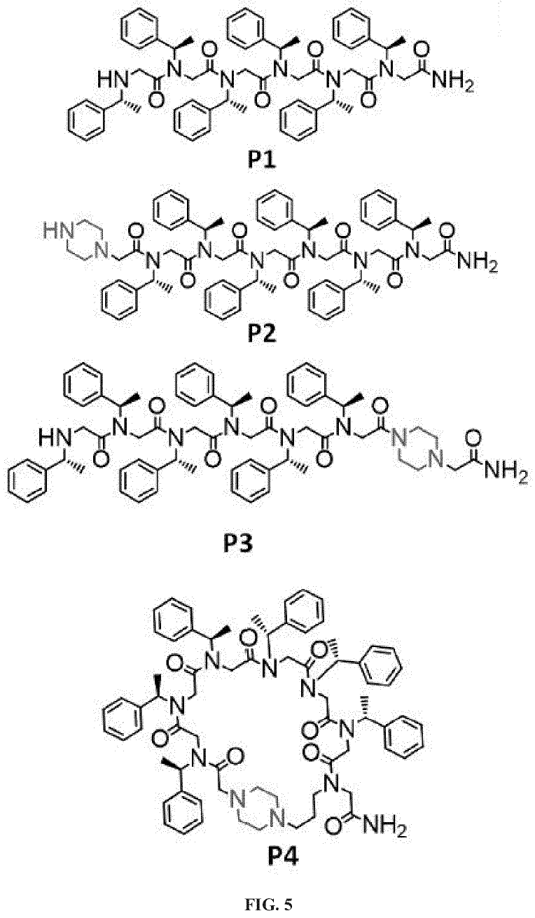

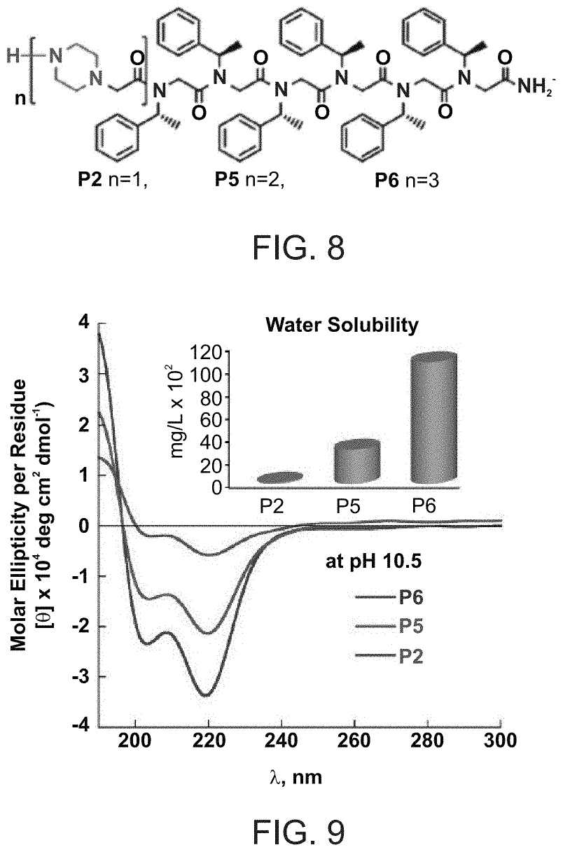

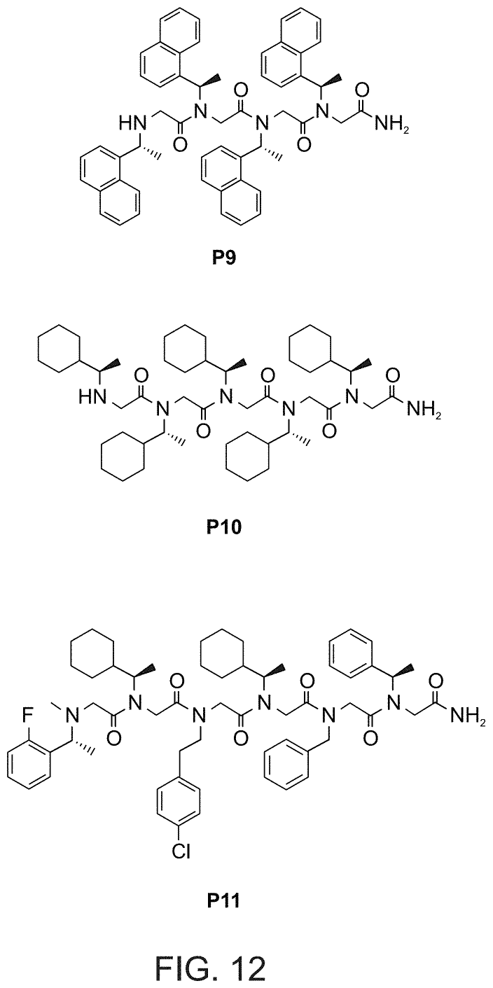

Referring now to the drawings, FIG. 1 depicts a piperazine-containing peptoid (P2) according to some embodiments of the invention, along with peptoids modified by alternative polar groups. FIGS. 5 and 8 depict additional piperazine-containing peptoids (P2, P3, P4, P5, P6) according to some embodiments of the invention, along with the structure of the corresponding unmodified peptoid (P1, in FIG. 5). FIG. 3 schematically describes synthesis of piperazine-containing peptoids. FIG. 12 depicts additional types of peptoids which were modified according to some embodiments of the invention.

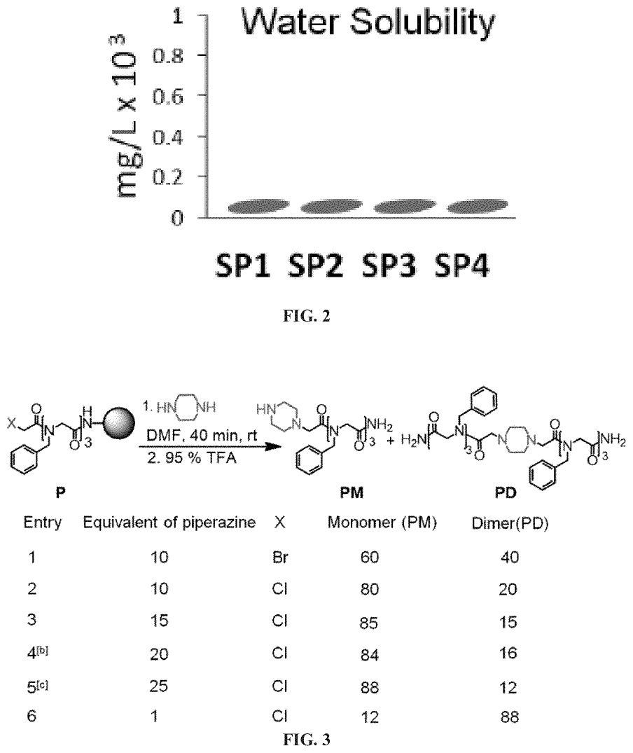

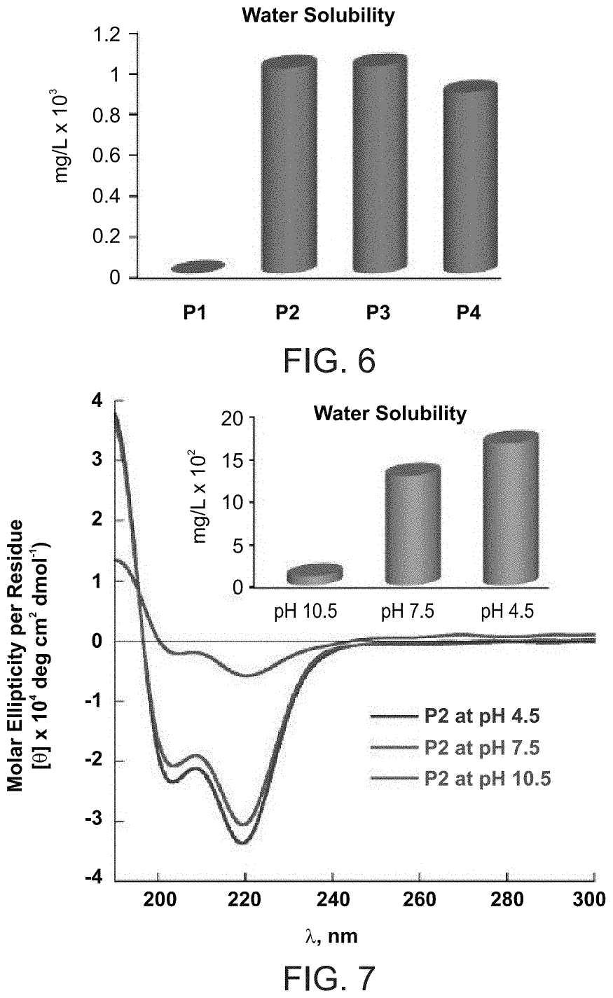

FIG. 6 shows that incorporation of a piperazine residue in a hydrophobic peptoid considerably enhances water solubility of the peptoid. FIG. 2 shows that that incorporation of alternative polar groups (as depicted in FIG. 1) in a hydrophobic peptoid was considerably less effective than piperazine incorporation at enhancing water solubility. FIG. 9 shows that water-solubility of piperazine-containing peptoids is correlated with the number of piperazine residues. FIG. 7 shows that water-solubility of piperazine-containing peptoids is pH-dependent.

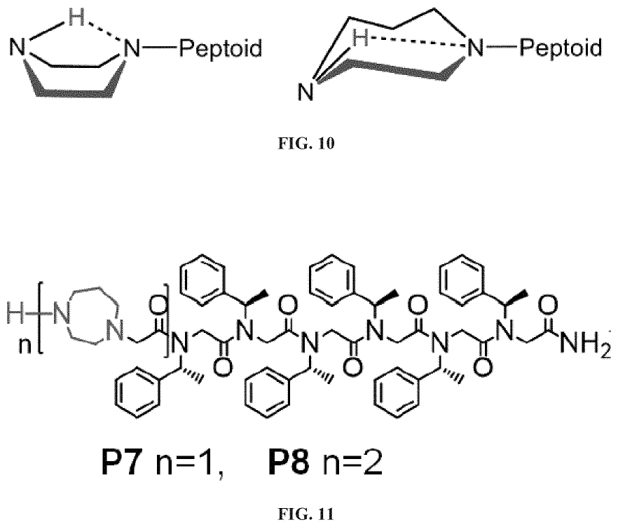

FIG. 11 depicts homopiperazine-containing peptoids (P7, P8) according to some embodiments of the invention. FIG. 10 depicts configurations of piperazine and homopiperazine which may explain why homopiperazine was more effective than piperazine at increasing water solubility of peptoids.

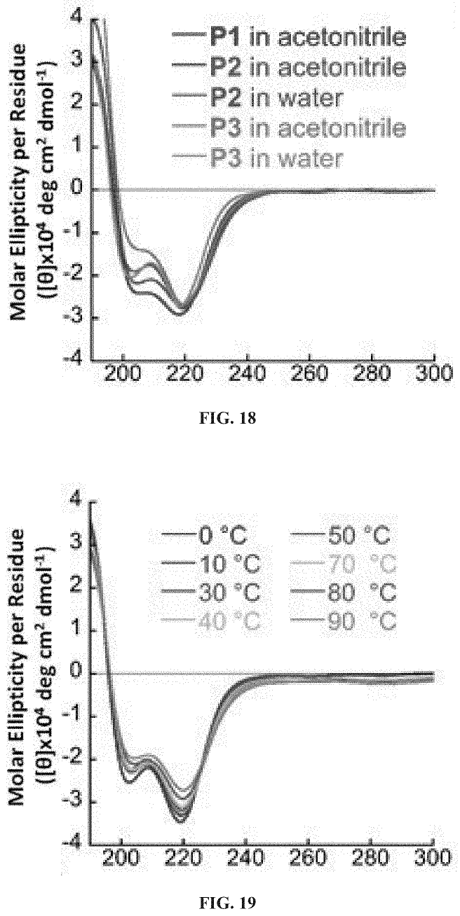

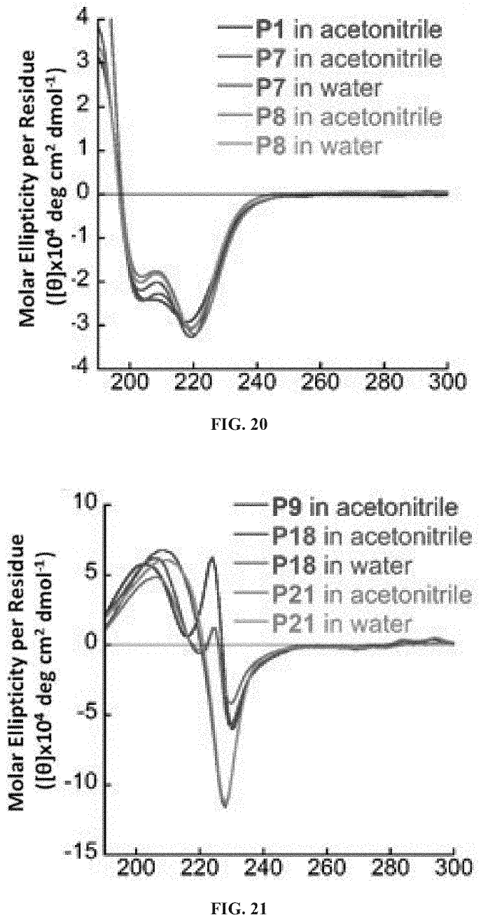

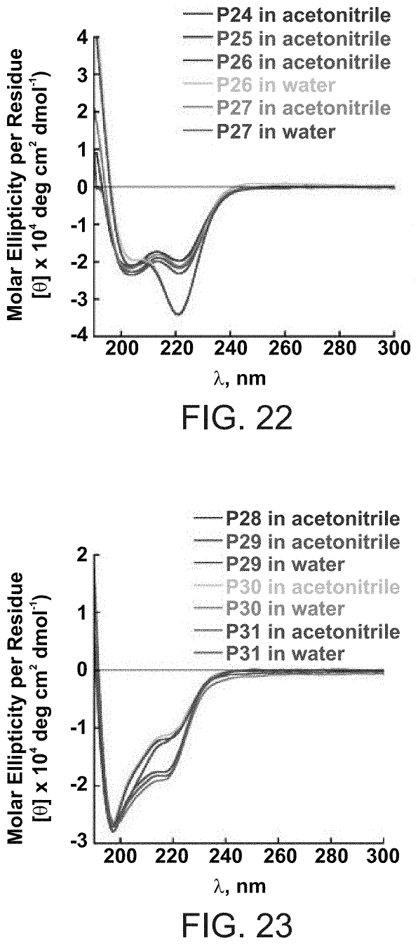

FIGS. 18 and 20 show that the hydrophobic peptoid P1 exhibits a characteristic helical secondary structure in acetonitrile, as determined by circular dichroism spectroscopy, and that corresponding water soluble modified peptoids (P2, P3, P7, P8) exhibit similar secondary structure in both acetonitrile and water. FIG. 19 shows that the water soluble modified peptoid secondary structure is stable in water at a broad range of temperatures (0 to 90° C.). FIG. 21 shows that the helical secondary structure of a hydrophobic peptoid was stabilized upon incorporation of piperazine groups. FIGS. 22 and 23 show that incorporation of piperazine groups facilitates secondary structure formation of β-peptoids and azapeptoids, respectively, in water.

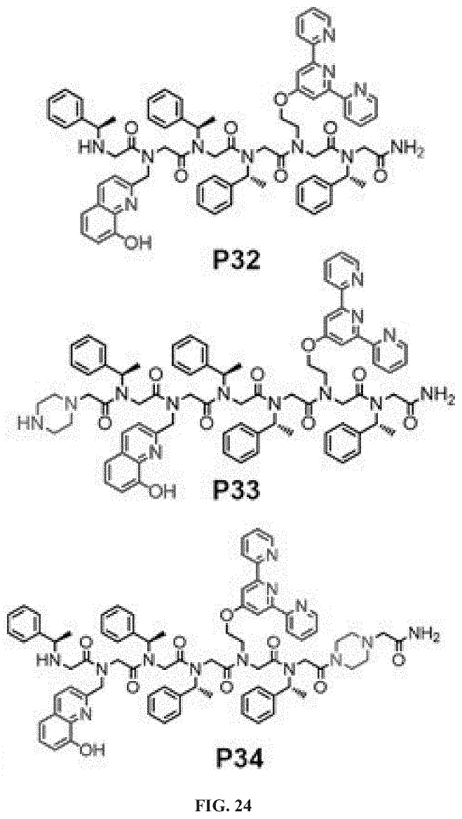

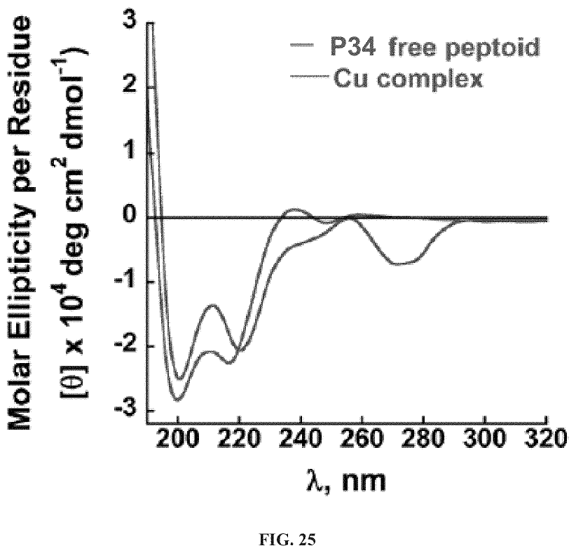

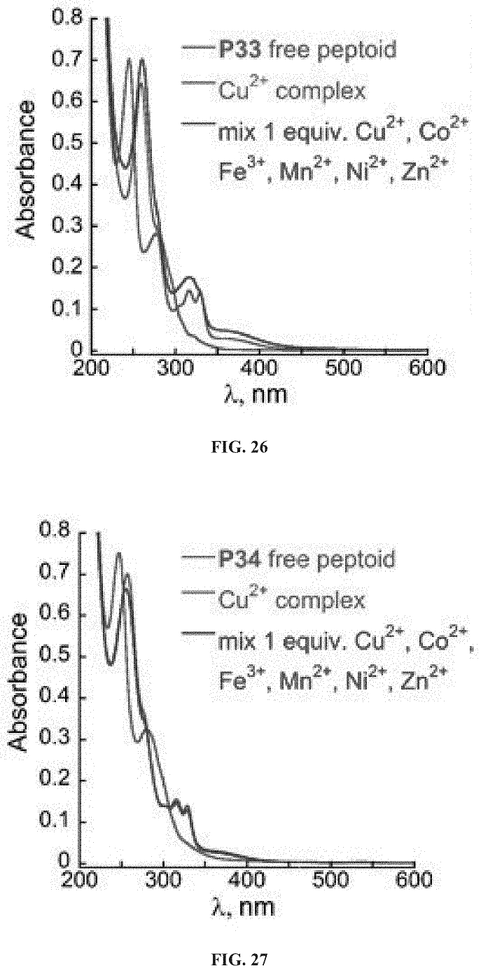

FIG. 24 depicts a water-insoluble copper-binding peptoid (P32) as well as exemplary water-soluble peptoids (P33, P34) obtained by modifying the water-insoluble peptoid by incorporating a piperazine group therein according to some embodiments of the invention. FIG. 25 shows that exemplary copper-binding peptoids retain their native secondary structure upon modification with piperazine. FIGS. 26 and 27 show that the water-soluble modified peptoids retain the ability to selectively bind copper ions.

These results indicate that hydrophobic peptoids can be modified so as to be dissolvable in water (e.g., water soluble) while substantially retaining the peptoid structure and functionality.

According to an aspect of some embodiments of the invention, there is provided a peptoid comprising a plurality of N-substituted glycine residues and/or N-substituted β-alanine (3-aminopropanoate) residues, the peptoid further comprising at least one heteroalicyclic residue, as described herein.

Herein, the term “peptoid” refers to a compound comprising a plurality of amino acid residues linked by amide bonds, wherein at least 50% of the amino acid residues are N-substituted glycine residues and/or N-substituted β-alanine (3-aminopropanoate) residues. Thus, the amino acid residues of the peptoid may optionally consist of N-substituted glycine residues and/or N-substituted β-alanine residues, or alternatively, may comprise N-substituted glycine residues and/or N-substituted β-alanine residues in combination with one or more amino acid residue which is not N-substituted (i.e., a nitrogen atom of a backbone amide group is attached, inter alia, to a hydrogen atom), for example, a residue of a standard amino acid (other than proline), e.g., a non-substituted glycine residue, and/or a residue of a β-amino acid, e.g., a non-substituted β-alanine residue.

It is to be appreciated that heteroalicyclic residues (according to the general formula described herein) are not considered herein as “amino acid residues” according to the abovementioned definition of the term “peptoid”; such that a number of heteroalicyclic residues (according to the general formula described herein) does not affect the percentage of amino acid residues in a compound which are N-substituted glycine residues and/or N-substituted β-alanine residues, and thus does not affect whether a given compound meets the abovementioned definition of the term “peptoid”.

According to some of any of the embodiments described herein, the plurality of amino acid residues comprises at least 3 amino acid residues (optionally at least 3 N-substituted glycine residues and/or N-substituted β-alanine residues) linked by amide bonds. In some embodiments, the plurality of amino acid residues comprises at least 4 amino acid residues (optionally at least 4 N-substituted glycine residues and/or N-substituted β-alanine residues) linked by amide bonds. In some embodiments, the plurality of amino acid residues comprises at least 5 amino acid residues (optionally at least 5 N-substituted glycine residues and/or N-substituted β-alanine residues) linked by amide bonds. In some embodiments, the plurality of amino acid residues comprises at least 6 amino acid residues (optionally at least 6 N-substituted glycine residues and/or N-substituted β-alanine residues) linked by amide bonds.

Heteroalicyclic Residue:

The peptoid according to any of the embodiments described herein comprises at least one heteroalicyclic residue, having a general formula:

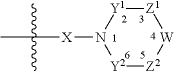

wherein:

X is —C(═O)—CR₁R₂— or —NR_{16—} or absent;

Y¹ is CR₃R₄ or C═O;

Y² is CR₅R₆ or C═O;

Z¹ is CR₇R₈ or C═O; and

Z² is CR₉R₁₀ or CR₁₁R₁₂—CR₁₃R₁₄.

W is optionally NR₁₅, S(═O), S(═O)2, S or O, in which case the residue is attached to the remainder of the peptoid via the X moiety (or, when X is absent, via the adjacent nitrogen atom); or alternatively, W is N—, that is, a nitrogen atom attached by a covalent bond to another portion of the peptoid; such that the heteroalicyclic residue is attached to a portion of the peptoid via a covalent bond with the nitrogen atom N— (and optionally attached to another portion of the peptoid via the X moiety, or, when X is absent, via the adjacent nitrogen atom).

For convenience, the positions of the ring are arbitrarily numbered as 1, 2 (the position of Y¹), 3 (the position of Z¹), 4 (the position of W), 5 (the position of Z²) and 6 (the position of Y²).

R₁-R₁₄ are each independently hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, phosphate, phosphonyl, phosphinyl, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, hydrazine, or amino; and

R₁₅ (attached to a nitrogen atom in W) and Rib (attached to a nitrogen atom in X) are each independently hydrogen, alkyl, alkenyl, alkynyl, cycloalkyl, aryl, heteroaryl, heteroalicyclic, carbonyl, thiocarbonyl, C-amido, or C-carboxy (as these terms are defined herein).

Alternatively, any two of R₁-R₁₆ (which are present in the same compound) optionally together form a 5-membered, 6-membered or 7-membered alicyclic or heteroalicyclic ring. In some embodiments of any of the embodiments described herein, only R₃-R₁₄ may optionally form a 5-, 6- or 7-membered alicyclic or heteroalicyclic ring, optionally a heteroalicyclic ring. In some embodiments, a 5-, 6- or 7-membered heteroalicyclic ring (optionally a 7-membered ring) may optionally be formed from R₃ together with R₅ (i.e., thereby forming a ring which includes Y¹, Y² and the nitrogen atom between Y¹ and Y²), and/or from R₇ together with R₉ (i.e., thereby forming a ring which includes Z¹, Z² and W).

In some embodiments of any of the embodiments described herein, Z² is CR₉Rio. In such embodiments, the heteroalicyclic ring comprises a 6-membered ring with nitrogen atoms at 1 and 4 positions thereof. Such a ring is referred to herein interchangeably as a “piperazine”.

In some embodiments of any of the embodiments wherein Z² is CR₉R₁₀, R₃-R₁₀ are each hydrogen (e.g., such that the piperazine ring is unsubstituted at the carbon atoms thereof). In some embodiments of any of the embodiments described herein, Z² is CR₁₁R₁₂CR₁₃R_{14.}

In such embodiments, the heteroalicyclic ring comprises a 7-membered ring with nitrogen atoms at 1 and 4 positions thereof (which may be alternatively numbered as 1 and 5 positions thereof). Such a ring is referred to herein interchangeably as a “homopiperazine”.

In some embodiments of any of the embodiments wherein Z² is CR₁₁R₁₂CR₁₃R₁₄, R₃-R₈ and R₁₁-R₁₄ are each hydrogen (e.g., such that the homopiperazine ring is unsubstituted at the carbon atoms thereof).

In some embodiments of any of the embodiments described herein, Y¹ is CR₃R₄ (i.e., Y¹ is not C═O).

In some embodiments of any of the embodiments described herein, Y² is CR₅R₆ (i.e., Y² is not C═O).

In some embodiments of any of the embodiments described herein, Y¹ is CR₃R₄ and Y² is CR₅R₆ (i.e., neither Y¹ nor Y² is C═O).

In some embodiments of any of the embodiments described herein, Z¹ is CR₇R₈ (i.e., Z¹ is not C═O). In some such embodiments, Z¹ is CR₇R₈ and Y¹ is CR₃R₄ (i.e., neither Z¹ nor Y¹ is C═O). In some such embodiments, Z¹ is CR₇R₈ and Y² is CR₅R₆ (i.e., neither Z¹ nor Y¹ is C═O).

In some embodiments, Z¹ is CR₇R₈, Y¹ is CR₃R₄ and Y² is CR₅R₆ (i.e., none of Z¹, Y¹ or Y² is C═O).

In some embodiments of any of the embodiments described herein, X is —C(═O)—CR₁R₂— (i.e., X is not —NR_{16—}).

In some embodiments of any of the embodiments described herein, R₃-R₁₄ are each independently hydrogen, alkyl (e.g., C₁₋₄-alkyl), aryl (e.g., substituted or unsubstituted phenyl), or C-amido (e.g., tert-butylaminocarbonyl), or alternatively, R₃ and R₅ (and/or R₇ and R₉) together form a 5-, 6- or 7-membered heteroalicyclic ring (e.g., an unsubstituted 7-membered azepane ring).

In some embodiments of any of the embodiments described herein, R₁-R₁₄ are each independently hydrogen, C₁₋₄-alkyl (e.g., methyl, trifluoromethyl), C₂₋₄-alkenyl, C₂₋₄-alkynyl, phenyl (e.g., 3-methoxyphenyl), C₁₋₄-alkoxy, C₁₋₄-thioalkoxy, C₁₋₄-carbonyl, C₁₋₄-thiocarbonyl, C₁₋₄—O-carboxy, nitro, —S(═O)₂OH, —O—S(═O)₂OH, —S(═O)₂NH₂, —C(═O)NH₂ or —C(═O)OH, or alternatively, any two of R₁-R₁₄ together form a 5-, 6- or 7-membered heteroalicyclic ring. In some such embodiments, R₁-R₁₄ are each independently hydrogen or C₁₋₄-alkyl. In some such embodiments, R₁-R₁₄ are each independently hydrogen or methyl (optionally unsubstituted methyl).

In some embodiments of any of the embodiments described herein, R₁₅-R₁₆ are each independently hydrogen, C₁₋₄-alkyl (e.g., methyl), C₂₋₄-alkenyl, C₂₋₄-alkynyl, phenyl (e.g., unsubstituted phenyl), C₁₋₄-carbonyl, C₁₋₄-thiocarbonyl, C₁₋₄—O-carboxy, nitro, —S(═O)₂OH, —O—S(═O)₂OH, —S(═O)₂NH₂, —C(═O)NH₂ or —C(═O)OH. In some such embodiments, R₁₅-R₁₆ are each independently hydrogen or C₁₋₄-alkyl. In some such embodiments, R₁₅-R₁₆ are each independently hydrogen or methyl (optionally unsubstituted methyl).

In some embodiments of any of the embodiments described herein, R₁-R₁₆ are each hydrogen or methyl (e.g., unsubstituted methyl or trifluoromethyl). In some embodiments, R₁-R₁₆ are each hydrogen or unsubstituted methyl.

In some embodiments of any of the embodiments described herein, R₁ and R₂ are each hydrogen.

In some embodiments of any of the embodiments described herein, R₁₆ is hydrogen. In some embodiments, R₁₆, R₁ and R₂ are each hydrogen. In some embodiments, R₁₆ and R₁-R₁₄ are each hydrogen. In some embodiments, R₁-R₁₆ are each hydrogen.

In some embodiments of any of the embodiments described herein, at least one heteroalicyclic residue (according to any of the respective embodiments described herein) is a terminal residue.

Herein, the term “terminal residue” refers to a residue which is attached to the remainder of the peptoid via one covalent bond. A terminal residue may optionally comprise W being NR₁₅ (optionally NH), S(═O), S(═O)₂, S or O; or alternatively, W is a nitrogen atom attached by a covalent bond to another portion of the peptoid (N—), and X (or, when X is absent, the adjacent nitrogen atom) is not attached to another residue, but rather to, for example, hydrogen (e.g., when X is absent), hydroxy (e.g., such that X is a part of a HOC(═O)—CR₁R₂— group) or amino (e.g., such that X is a part of a H₂NC(═O)—CR₁R₂— group).

In some embodiments of any of the embodiments described, W is NR₁₅ or O, or a nitrogen atom attached by a covalent (e.g., amide) bond to another portion of the peptoid. In some embodiments, W is NR₁₅ or a nitrogen atom attached by a covalent (e.g., amide) bond to another portion of the peptoid.

In some embodiments of any of the embodiments described herein wherein W is NR₁₅, R₁₅ is hydrogen, alkyl (optionally C₁₋₄-alkyl), aryl or C-carboxy (e.g., a C-carboxy ester). In exemplary embodiments, R₁₅ is hydrogen, phenyl, methyl, 2-hydroxyethyl, or tert-butyloxycarbonyl (an exemplary C-carboxy ester, also known as “Boc”).

In some embodiments of any of the embodiments described herein wherein W is a nitrogen atom attached by a covalent bond to another portion of the peptoid, the nitrogen atom (represented by W) is optionally attached to a carbonyl group (e.g., of an amino acid residue), thereby forming an amide bond.

Alternatively, the nitrogen atom (represented by W) is optionally attached to a carbon atom of a carboxylic acid residue (e.g., at an a or β position relative to the carboxylic acid group), thereby forming an amino acid residue (e.g., an α-amino acid residue such as a glycine residue, or a β-amino acid residue such as a β-alanine residue).

Alternatively, the nitrogen atom (represented by W) is optionally attached to a carbon atom in a side chain of an N-substituted residue.

In some embodiments of any of the embodiments described herein, at least one heteroalicyclic residue (according to any of the respective embodiments described herein) is incorporated into the peptoid backbone as a terminal or non-terminal residue, by being directly attached to an amino acid residue (e.g., N-substituted residue) via an amide bond to a backbone nitrogen atom or a backbone carbonyl of the amino acid residue (i.e., the nitrogen atom or carbonyl of a glycine or β-alanine residue, rather than a nitrogen atom or carbonyl of a side chain thereof).

Such an amide bond may optionally be formed from a nitrogen atom of the heteroalicyclic residue (e.g., wherein W is a nitrogen atom and/or the nitrogen atom adjacent to X when X is absent) being attached to a backbone carbonyl of an amino acid residue (e.g., at a C-terminus of the peptoid). Alternatively or additionally, such an amide bond may optionally be formed from a carbonyl of the heteroalicyclic residue (e.g., wherein X is —C(═O)—CR₁R₂) being attached to a backbone nitrogen atom of an amino acid residue (e.g., at an N-terminus of the peptoid, or at a non-terminal portion of the backbone wherein another amide bond is formed from a nitrogen atom of the heteroalicyclic residue being attached to a backbone carbonyl, as described herein).

In some embodiments of any of the embodiments described herein, the peptoid comprises a single heteroalicyclic residue, at a terminal position or a non-terminal position (according to any of the respective embodiments described herein).

In alternative embodiments of any of the embodiments described herein, the peptoid comprises at least two heteroalicyclic residues (according to any of the respective embodiments described herein), optionally two or three such heteroalicyclic residues. Each of the heteroalicyclic residues may independently be at a terminal position or a non-terminal position.

In some embodiments of any of the embodiments described herein, at least two (e.g., two or three) heteroalicyclic residues are attached to one another. In some embodiments, such attached heteroalicyclic residues are at a terminal position of the peptoid (e.g., wherein one of the heteroalicyclic residues is a terminal residue as defined herein, and another heteroalicyclic residue(s) is attached to the terminal residue by one covalent bond and to the remainder of the peptoid via another covalent bond). Exemplary peptoids with two or three attached heteroalicyclic residues at a terminal position are described in the Examples section and Figures herein.

In some embodiments of any of the embodiments described herein, a ratio of a number of heteroalicyclic residues (according to any of the respective embodiments described herein) in the peptoid to a total number of N-substituted glycine and N-substituted β-alanine residues in the peptoid is no more than 1:1 (heteroalicyclic: N-substituted glycine/β-alanine), that is, the number of heteroalicyclic residues does not exceed the total number of N-substituted glycine and N-substituted β-alanine residues. In some embodiments, the ratio of the number of heteroalicyclic residues (according to any of the respective embodiments described herein) to the total number of N-substituted glycine and N-substituted β-alanine residues is in a range of from 0.1:1 to 1:1 (heteroalicyclic: N-substituted glycine/β-alanine).

In some embodiments of any of the embodiments described herein, a ratio of heteroalicyclic residue weight (according to any of the respective embodiments described herein) to total weight of the peptoid (including, inter alia, weight of heteroalicyclic residue(s)) is at least one heteroalicyclic residue per 750 Da of the total weight of the peptoid. In some embodiments, the ratio is at least 1.5 heteroalicyclic residue per 750 Da of the total weight of the peptoid (i.e., at least one heteroalicyclic residue per 500 Da of the total weight of the peptoid).

In some embodiments of any of the embodiments described herein, a ratio of heteroalicyclic residue weight (according to any of the respective embodiments described herein) to total weight of the peptoid (including, inter alia, weight of heteroalicyclic residue(s)) is no more than two heteroalicyclic residues per 750 Da of the total weight of the peptoid (i.e., no more than one heteroalicyclic residue per 375 Da of the total weight of the peptoid). In some embodiments, the ratio is no more than at least 1.5 heteroalicyclic residue per 750 Da of the total weight of the peptoid.

In some embodiments of any of the embodiments described herein, a ratio of heteroalicyclic residue weight (according to any of the respective embodiments described herein) to total weight of the peptoid is in a range of from one to two heteroalicyclic residues per 750 Da of the total weight of the peptoid.

N-substituted Residues:

In some embodiments of any of the embodiments described herein, at least a portion of the N-substituted glycine and/or N-substituted β-alanine residues of the peptoid comprise a side chain (i.e., a substituent indicated by the term “N-substituted”) which comprises an aryl, heteroaryl, cycloalkyl or heteroalicyclic moiety. In some embodiments, at least 25% of the N-substituted (glycine and/or β-alanine) residues comprise such a side chain. In some embodiments, at least 50% of the N-substituted (glycine and/or β-alanine) residues comprise such a side chain. In some embodiments, at least 75% of the N-substituted (glycine and/or β-alanine) residues comprise such a side chain. In some embodiments, each of the N-substituted (glycine and/or β-alanine) residues comprises such a side chain.

The side chain(s) of a peptoid may be any type of substituent, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphate, phosphonyl, phosphinyl, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, as these terms are defined herein.

In some embodiments of any of the embodiments described herein, the side chain of at least a portion of the N-substituted glycine and/or N-substituted β-alanine residues consists of a substituted or non-substituted aryl, heteroaryl, cycloalkyl or heteroalicyclic. In some embodiments, at least 25% of the N-substituted (glycine and/or β-alanine) residues comprise such a side chain. In some embodiments, at least 50% of the N-substituted (glycine and/or β-alanine) residues comprise such a side chain. In some embodiments, at least 75% of the N-substituted (glycine and/or β-alanine) residues comprise such a side chain. In some embodiments, each of the N-substituted (glycine and/or β-alanine) residues comprises such a side chain.

In some embodiments of any of the embodiments described herein, the side chain of at least a portion of the N-substituted glycine residues and/or N-substituted β-alanine residues consists of an alkyl (optionally a C₁₋₄-alkyl, e.g., methyl, ethyl) substituted by at least one (substituted or non-substituted) aryl, heteroaryl, cycloalkyl or heteroalicyclic (and optionally by one or more additional substituent). In some embodiments, at least 25% of the N-substituted (glycine and/or β-alanine) residues comprise such a side chain. In some embodiments, at least 50% of the N-substituted (glycine and/or β-alanine) residues comprise such a side chain. In some embodiments, at least 75% of the N-substituted (glycine and/or β-alanine) residues comprise such a side chain. In some embodiments, each of the N-substituted (glycine and/or β-alanine) residues comprises such a side chain.

In some embodiments of any of the embodiments described herein, the side chain of at least a portion of the N-substituted glycine residues and/or N-substituted β-alanine residues consists of an alkyl substituted by at least one (substituted or non-substituted) aryl, heteroaryl, cycloalkyl or heteroalicyclic (and optionally by one or more additional substituent) at a 1 position or 2-position of the alkyl, for example, at the 1-position of a methyl or a 1-position or 2-position of an ethyl. In some embodiments, at least 25% of the N-substituted (glycine and/or β-alanine) residues comprise such a side chain. In some embodiments, at least 50% of the N-substituted (glycine and/or β-alanine) residues comprise such a side chain. In some embodiments, at least 75% of the N-substituted (glycine and/or β-alanine) residues comprise such a side chain. In some embodiments, each of the N-substituted (glycine and/or β-alanine) residues comprises such a side chain.

In some embodiments of any of the embodiments described herein, the side chain of at least a portion of the N-substituted glycine residues and/or N-substituted β-alanine residues is chiral. In some embodiments, at least 25% of the N-substituted (glycine and/or β-alanine) residues comprise a chiral side chain. In some embodiments, at least 50% of the N-substituted (glycine and/or β-alanine) residues comprise a chiral side chain. In some embodiments, at least 75% of the N-substituted (glycine and/or β-alanine) residues comprise a chiral side chain. In some embodiments, each of the N-substituted (glycine and/or β-alanine) residues comprises a chiral side chain.

Examples of a chiral side chain include, without limitation, alkyl substituted by aryl, heteroaryl, cycloalkyl or heteroalicyclic at a 1-position of ethyl or isobutyl, at a 1- or 2-position of n-propyl or butan-2-yl, or at a 1-, 2- or 3-position of n-butyl. In some embodiments, the chiral side chain as S chirality.

Examples of aryl and heteroaryl groups which may be comprised by side chains incorporated in an N-substituted residue (e.g., side chains consisting of alkyl substituted by the aryl or heteroaryl group) include, for example, unsubstituted phenyl; halo-substituted phenyl, such as chlorophenyl (e.g., 4-chlorophenyl) and/or fluorophenyl (e.g., 2-fluorophenyl); sulfonate-substituted phenyl (e.g., 4-sulfophenyl); naphthyl (e.g., naphthalen-l-yl); hydroxyquinolinyl (e.g., 8-hydroxyquinolin-2-yl); and terpyridinyl (e.g., (2,2′:6′,2″)terpyridin-4′-yl).

Cyclohexyl is an exemplary cycloalkyl group which may be comprised by side chains incorporated in an N-substituted residue (e.g., side chains consisting of alkyl substituted by the cycloalkyl moiety.

Examples of a side chains which may be incorporated in an N-substituted residue include, for example, phenylmethyl, chlorophenylmethyl, fluorophenylmethyl, sulfophenylmethyl (e.g., 4-sulfophenylmethyl, a.k.a., p-sulfobenzyl), phenylethyl (e.g., 1-phenylethyl), chlorophenylethyl (e.g., 2-(4-chlorophenyl)ethyl), fluorophenylethyl (e.g., 1-(2-fluorophenyl)ethyl), sulfophenylethyl, naphthylmethyl, naphthylethyl (e.g., 1-(naphthalen-1-yl)ethyl), cyclohexylmethyl, cyclohexylethyl (e.g., 1-cyclohexylethyl), hydroxyquinolinylmethyl (e.g., 8-hydroxyquinolin-2-ylmethyl), hydroxyquinolinylethyl, terpyridinyloxyethyl (e.g., 2-((2,2′:6′,2″)terpyridin-4′-yloxy)ethyl), and alkoxycarbonylamino (e.g., ethoxycarbonylamino).

Peptoid Water-solubility and Aqueous Solutions:

According to embodiments of the present invention, peptoids according to some embodiments of the invention are dissolvable in an aqueous solution (e.g., water). By “dissolvable” it is meant that the peptoid is capable of being dissolved in an aqueous solution (e.g., water).

As exemplified in the Examples section herein, peptoids according to some embodiments of the invention exhibit considerable water-solubility.

In some embodiments of any of the embodiments described herein, a water-solubility of the peptoid is at least 100 mg/liter. In some embodiments, a water-solubility of the peptoid is at least 200 mg/liter. In some embodiments, a water-solubility of the peptoid is at least 300 mg/liter. In some embodiments, a water-solubility of the peptoid is at least 400 mg/liter. In some embodiments, a water-solubility of the peptoid is at least 500 mg/liter.

As further exemplified in the Examples section herein, the water-solubility of a peptoid comprising at least one heteroalicyclic residue (according to some embodiments of the invention) may be considerable greater than a water-solubility of a corresponding peptoid lacking the at least one heteroalicyclic residue, which may be, for example, substantially water-insoluble.

Herein, a “corresponding peptoid” (lacking at least one heteroalicyclic residue) in the context of a given peptoid having at least one alicyclic residue (according to any of the respective embodiments described herein) refers to a peptoid wherein each heteroalicyclic residue (or sequence of two heteroalicyclic residues attached to one another) of the given peptoid (according to any of the respective embodiments described herein) is replaced by a hydrogen atom (e.g., wherein the heteroalicyclic residue(s) is a terminal residue(s) according to any of the respective embodiments described herein) or by a covalent bond (e.g., a covalent bond linking N-substituted residues according to any of the respective embodiments described herein).

In some embodiments of any of the embodiments described herein, a water-solubility of the corresponding peptoid (as defined herein) lacking a heteroalicyclic moiety is less than 100 mg/liter. In some such embodiments, a water-solubility of the peptoid (comprising at least one heteroalicyclic moiety) is at least 200 mg/liter. In some embodiments, a water-solubility of the peptoid is at least 300 mg/liter. In some embodiments, a water-solubility of the peptoid is at least 400 mg/liter. In some embodiments, a water-solubility of the peptoid is at least 500 mg/liter.

In some embodiments of any of the embodiments described herein, a water-solubility of the corresponding peptoid (as defined herein) lacking a heteroalicyclic moiety is less than 50 mg/liter. In some such embodiments, a water-solubility of the peptoid (comprising at least one heteroalicyclic moiety) is at least 100 mg/liter. In some embodiments, a water-solubility of the peptoid is at least 200 mg/liter. In some embodiments, a water-solubility of the peptoid is at least 300 mg/liter. In some embodiments, a water-solubility of the peptoid is at least 400 mg/liter. In some embodiments, a water-solubility of the peptoid is at least 500 mg/liter.

In some embodiments of any of the embodiments described herein, a water-solubility of the corresponding peptoid (as defined herein) lacking a heteroalicyclic moiety is less than 20 mg/liter. In some such embodiments, a water-solubility of the peptoid (comprising at least one heteroalicyclic moiety) is at least 100 mg/liter. In some embodiments, a water-solubility of the peptoid is at least 200 mg/liter. In some embodiments, a water-solubility of the peptoid is at least 300 mg/liter. In some embodiments, a water-solubility of the peptoid is at least 400 mg/liter. In some embodiments, a water-solubility of the peptoid is at least 500 mg/liter.

In some embodiments of any of the embodiments described herein, a water-solubility of the corresponding peptoid (as defined herein) lacking a heteroalicyclic moiety is less than 10 mg/liter. In some such embodiments, a water-solubility of the peptoid (comprising at least one heteroalicyclic moiety) is at least 100 mg/liter. In some embodiments, a water-solubility of the peptoid is at least 200 mg/liter. In some embodiments, a water-solubility of the peptoid is at least 300 mg/liter. In some embodiments, a water-solubility of the peptoid is at least 400 mg/liter. In some embodiments, a water-solubility of the peptoid is at least 500 mg/liter.

In some embodiments of any of the embodiments described herein, a water-solubility of the corresponding peptoid (as defined herein) lacking a heteroalicyclic moiety is less than 5 mg/liter. In some such embodiments, a water-solubility of the peptoid (comprising at least one heteroalicyclic moiety) is at least 100 mg/liter. In some embodiments, a water-solubility of the peptoid is at least 200 mg/liter. In some embodiments, a water-solubility of the peptoid is at least 300 mg/liter. In some embodiments, a water-solubility of the peptoid is at least 400 mg/liter. In some embodiments, a water-solubility of the peptoid is at least 500 mg/liter.

In some embodiments of any of the embodiments described herein, a water-solubility of the corresponding peptoid (as defined herein) lacking a heteroalicyclic moiety is less than 2 mg/liter. In some such embodiments, a water-solubility of the peptoid (comprising at least one heteroalicyclic moiety) is at least 100 mg/liter. In some embodiments, a water-solubility of the peptoid is at least 200 mg/liter. In some embodiments, a water-solubility of the peptoid is at least 300 mg/liter. In some embodiments, a water-solubility of the peptoid is at least 400 mg/liter. In some embodiments, a water-solubility of the peptoid is at least 500 mg/liter.

In some embodiments of any of the embodiments described herein, a water-solubility of the corresponding peptoid (as defined herein) lacking a heteroalicyclic moiety is less than 1 mg/liter. In some such embodiments, a water-solubility of the peptoid (comprising at least one heteroalicyclic moiety) is at least 100 mg/liter. In some embodiments, a water-solubility of the peptoid is at least 200 mg/liter. In some embodiments, a water-solubility of the peptoid is at least 300 mg/liter. In some embodiments, a water-solubility of the peptoid is at least 400 mg/liter. In some embodiments, a water-solubility of the peptoid is at least 500 mg/liter.

In some embodiments of any of the embodiments described herein, a water-solubility of the corresponding peptoid (as defined herein) lacking a heteroalicyclic moiety is less than 0.5 mg/liter. In some such embodiments, a water-solubility of the peptoid (comprising at least one heteroalicyclic moiety) is at least 100 mg/liter. In some embodiments, a water-solubility of the peptoid is at least 200 mg/liter. In some embodiments, a water-solubility of the peptoid is at least 300 mg/liter. In some embodiments, a water-solubility of the peptoid is at least 400 mg/liter. In some embodiments, a water-solubility of the peptoid is at least 500 mg/liter.

In some embodiments of any of the embodiments described herein, a water-solubility of the corresponding peptoid (as defined herein) lacking a heteroalicyclic moiety is less than 0.2 mg/liter. In some such embodiments, a water-solubility of the peptoid (comprising at least one heteroalicyclic moiety) is at least 100 mg/liter. In some embodiments, a water-solubility of the peptoid is at least 200 mg/liter. In some embodiments, a water-solubility of the peptoid is at least 300 mg/liter. In some embodiments, a water-solubility of the peptoid is at least 400 mg/liter. In some embodiments, a water-solubility of the peptoid is at least 500 mg/liter.

In some embodiments of any of the embodiments described herein, a water-solubility of the corresponding peptoid (as defined herein) lacking a heteroalicyclic moiety is less than 0.1 mg/liter. In some such embodiments, a water-solubility of the peptoid (comprising at least one heteroalicyclic moiety) is at least 100 mg/liter. In some embodiments, a water-solubility of the peptoid is at least 200 mg/liter. In some embodiments, a water-solubility of the peptoid is at least 300 mg/liter. In some embodiments, a water-solubility of the peptoid is at least 400 mg/liter. In some embodiments, a water-solubility of the peptoid is at least 500 mg/liter.

In some embodiments of any of the embodiments described herein, a water-solubility of a peptoid (comprising at least one heteroalicyclic residue according to embodiments of the invention) is at least 150% of (i.e., 50% more than) a water solubility of a corresponding peptoid thereof (lacking any such heteroalicyclic residue(s)). In some embodiments, a water-solubility of the peptoid is at least 200% of (i.e., two-fold) a corresponding peptoid thereof. In some embodiments, a water-solubility of the peptoid is at least 300% of (i.e., 3-fold) a corresponding peptoid thereof. In some embodiments, a water-solubility of the peptoid is at least 500% of (i.e., 5-fold) a corresponding peptoid thereof. In some embodiments, a water-solubility of the peptoid is at least 1000% of (i.e., 10-fold) a corresponding peptoid thereof. In some embodiments, a water-solubility of the peptoid is at least 20-fold a corresponding peptoid thereof. In some embodiments, a water-solubility of the peptoid is at least 50-fold a corresponding peptoid thereof. In some embodiments, a water-solubility of the peptoid is at least 100-fold a corresponding peptoid thereof. In some embodiments, a water-solubility of the peptoid is at least 200-fold a corresponding peptoid thereof. In some embodiments, a water-solubility of the peptoid is at least 500-fold a corresponding peptoid thereof. In some embodiments, a water-solubility of the peptoid is at least 1000-fold a corresponding peptoid thereof. In some embodiments, a water-solubility of the peptoid is at least 2000-fold a corresponding peptoid thereof. In some embodiments, a water-solubility of the peptoid is at least 5000-fold a corresponding peptoid thereof. In some embodiments, a water-solubility of the peptoid is at least 10000-fold a corresponding peptoid thereof.

In some embodiments of any of the embodiments described herein, the peptoid is characterized by a helical structure in aqueous solution, e.g., the dissolved peptoid (in aqueous solution) has a defined secondary structure.

Without being bound by any particular theory, it is believed that a helical structure is associated with specific functional activity of many peptoids, and that the combination of water-solubility and helical structure facilitates provision of specific functional activity in aqueous environments, which has heretofore been difficult to obtain.

In some embodiments of any of the embodiments described herein, a corresponding peptoid (as defined herein) lacking the heteroalicyclic residue(s) (according to any of the respective embodiments described herein) is characterized by a helical structure in an organic solution, for example, in acetonitrile (e.g., the dissolved peptoid (in organics solution) has a defined secondary structure). In some embodiments, a helical structure of the corresponding peptoid in organic solution (e.g., in acetonitrile) is also present in the peptoid according to embodiments of the invention in aqueous solution.

A helical structure (of a peptoid) in solution (organic and/or aqueous solution) may optionally be determined by circular dichroism spectroscopy (e.g., according to procedures exemplified herein) and/or by NMR spectroscopy, using techniques known in the art.

According to an aspect of some embodiments of the invention, there is provided a composition comprising the peptoid described herein (according to any of the respective embodiments) and an aqueous liquid. In some such embodiments, the peptoid is soluble in the aqueous liquid, such that an aqueous liquid solution of the peptoid is formed. A pH of the aqueous liquid solution is optionally in a range of from 5 to 9, and optionally from 6 to 8, and optionally about 7.

Herein, the term “soluble” refers to an ability of a compound to dissolve in a liquid at a concentration (of dissolved material) of at least 1 mg/liter. It is to be appreciated that the term “soluble”, as used herein, does not encompass dispersion and/or suspension of a peptoid in the aqueous liquid, wherein the peptoid remains a separate phase from the aqueous liquid.

In some embodiments of any of the embodiments described herein, a concentration of the peptoid in the aqueous liquid is at least 3 mg/liter. In some embodiments, a concentration of the peptoid in the aqueous liquid is at least 10 mg/liter. In some embodiments, a concentration of the peptoid in the aqueous liquid is at least 30 mg/liter. In some embodiments, a concentration of the peptoid in the aqueous liquid is at least 100 mg/liter. In some embodiments, substantially all of the peptoid is dissolved in the aqueous liquid at the aforementioned concentration.

In some embodiments of any of the embodiments described herein relating to a composition, the composition is for use in binding a ligand, which is soluble in the aqueous liquid of the composition (e.g., binding the ligand in an aqueous environment).

According to an aspect of some embodiments of the invention, there is provided a method of binding a ligand, the method comprising contacting the ligand with a peptoid according to any of the respective embodiments described herein, the peptoid being capable of binding the ligand.

A ligand bound according to any of the respective embodiments according to any of the aspects described herein may be any ion and/or compound, for example, an inorganic ion chelated by the peptoid, a saccharide (e.g., a monosaccharide, disaccharide, or polysaccharide), or a polypeptide (e.g., a protein). In some embodiments, the ligand is a pharmacological target (e.g., a biomolecule), for example, wherein the peptoid is an agonist or antagonist of the ligand.

A ligand bound according to any of the respective embodiments according to any of the aspects described herein may be any ion and/or compound, for example, an inorganic ion chelated by the peptoid, a saccharide (e.g., a monosaccharide, disaccharide, or polysaccharide), or a polypeptide (e.g., a protein). In some embodiments, the ligand is a pharmacological target (e.g., a biomolecule), for example, wherein the peptoid is an agonist or antagonist of the ligand.

Without being bound by any particular theory, it is believed that some water-solubility may be advantageous in embodiments wherein a peptoid is intended for use within a largely aqueous physiological environment.

In some embodiments of any of the embodiments described herein relating to a peptoid and/or composition, the peptoid and/or composition is for use in treating a condition treatable by binding a ligand (e.g., a ligand known in the art as a pharmacological target for treatment of the condition), the peptoid being capable of binding the ligand. In some such embodiments, the peptoid is an agonist of the ligand. In some such embodiments, the peptoid is an antagonist of the ligand.

According to an aspect of some embodiments of the invention, there is provided a method of treating a peptoid and/or composition (according to any of the respective embodiments described herein) to the subject, wherein the condition is treatable by binding a ligand (e.g., a ligand known in the art as a pharmacological target for treatment of the condition), the peptoid being capable of binding the ligand. In some such embodiments, the peptoid is an agonist of the ligand. In some such embodiments, the peptoid is an antagonist of the ligand.

Determination of a peptoid structure capable of binding to a given ligand (e.g., pharmacological target) may be effected according to techniques known in the art, for example, by screening a library of peptoids (e.g., water-soluble peptoids according to any of the respective embodiments described herein), optionally prepared using combinatorial chemistry.

For a given use or method of a peptoid (e.g., binding a given ligand), a suitable peptoid comprising at least one heteroalicyclic according to embodiments described herein may optionally be selected based on a corresponding peptoid (as defined herein) lacking a heteroalicyclic residue, the corresponding peptoid (optionally being water-insoluble) being selected suitable for effecting a use or method (e.g., binding a given ligand), and modifying the corresponding peptoid so as to include at least one heteroalicyclic residue as described herein, for example, a number and species of heteroalicyclic residues sufficient to provide a peptoid characterized by a desired water solubility (as well as by a desired activity).

Additional Definitions and Information:

As used herein throughout, the term “alkyl” refers to any saturated aliphatic hydrocarbon including straight chain and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or non-substituted. When substituted, the substituent group can be, for example, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphate, phosphonyl, phosphinyl, oxo, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, as these terms are defined herein.

Herein, the term “alkenyl” describes an unsaturated aliphatic hydrocarbon comprise at least one carbon-carbon double bond, including straight chain and branched chain groups. Preferably, the alkenyl group has 2 to 20 carbon atoms. More preferably, the alkenyl is a medium size alkenyl having 2 to 10 carbon atoms. Most preferably, unless otherwise indicated, the alkenyl is a lower alkenyl having 2 to 4 carbon atoms. The alkenyl group may be substituted or non-substituted. Substituted alkenyl may have one or more substituents, whereby each substituent group can independently be, for example, cycloalkyl, alkynyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphate, phosphonyl, phosphinyl, oxo, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, as these terms are defined herein.

Herein, the term “alkynyl” describes an unsaturated aliphatic hydrocarbon comprise at least one carbon-carbon triple bond, including straight chain and branched chain groups. Preferably, the alkynyl group has 2 to 20 carbon atoms. More preferably, the alkynyl is a medium size alkynyl having 2 to 10 carbon atoms. Most preferably, unless otherwise indicated, the alkynyl is a lower alkynyl having 2 to 4 carbon atoms. The alkynyl group may be substituted or non-substituted. Substituted alkynyl may have one or more substituents, whereby each substituent group can independently be, for example, cycloalkyl, alkenyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphate, phosphonyl, phosphinyl, oxo, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, as these terms are defined herein.

A “cycloalkyl” group refers to a saturated on unsaturated all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphate, phosphonyl, phosphinyl, oxo, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, as these terms are defined herein. When a cycloalkyl group is unsaturated, it may comprise at least one carbon-carbon double bond and/or at least one carbon-carbon triple bond.

An “aryl” group refers to an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphate, phosphonyl, phosphinyl, oxo, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, as these terms are defined herein.

A “heteroaryl” group refers to a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphate, phosphonyl, phosphinyl, oxo, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, as these terms are defined herein.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or non-substituted. When substituted, the substituted group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphate, phosphonyl, phosphinyl, oxo, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, as these terms are defined herein. Representative examples are piperidine, piperazine, tetrahydrofuran, tetrahydropyran, morpholine and the like.

Herein, the terms “amine” and “amino” each refer to either a —NR′R″ group or a —N⁺R′R″R′″ group, wherein R′, R″ and R′″ are each hydrogen or a saturated or unsaturated hydrocarbon moiety (e.g., alkyl, alkenyl, alkynyl, cycloalkyl, or aryl, or one or more carbon atoms of a heteroaryl or heteroalicyclic), the hydrocarbon moiety being substituted or non-substituted. Optionally, R′, R″ and R′″ are hydrogen or alkyl comprising 1 to 4 carbon atoms. Optionally, R′ and R″ (and R′″, if present) are hydrogen. When substituted, the carbon atom of an R′, R″ or R″′ hydrocarbon moiety which is bound to the nitrogen atom of the amine is preferably not substituted by oxo, such that R′, R″ and R″′ are not (for example) carbonyl, C-carboxy or amide, as these groups are defined herein, except where indicated otherwise.

An “azide” group refers to a —N═N⁺=N⁻ group.

An “alkoxy” group refers to both an —O-alkyl and an —O-cycloalkyl group, as defined herein.

An “aryloxy” group refers to both an —O-aryl and an —O-heteroaryl group, as defined herein.

A “hydroxy” group refers to a —OH group.

A “thiohydroxy” or “thiol” group refers to a —SH group.

A “thioalkoxy” group refers to both an —S-alkyl group, and an —S-cycloalkyl group, as defined herein.

A “thioaryloxy” group refers to both an —S-aryl and an —S-heteroaryl group, as defined herein.

A “carbonyl” group refers to a —C(═O)—R′ group, where R′ is defined as hereinabove.

A “thiocarbonyl” group refers to a —C(═S)—R′ group, where R′ is as defined herein.

A “carboxyl”, “carboxylic” or “carboxylate” refers to both “C-carboxy” and “O-carboxy”.

A “C-carboxy” group refers to a —C(═O)—O—R′ groups, where R′ is as defined herein.

An “O-carboxy” group refers to an R′C(═O)—O— group, where R′ is as defined herein.

A “carboxylic acid” refers to a —C(═O)OH group, including the deprotonated ionic form and salts thereof.

An “oxo” group refers to a ═O group.

A “thiocarboxy” or “thiocarboxylate” group refers to both —C(═S)—O—R′ and —O—C(═S)R′ groups.

A “halo” group refers to fluorine, chlorine, bromine or iodine.

A “sulfinyl” group refers to an —S(═O)—R′ group, where R′ is as defined herein.

A “sulfonyl” group refers to an —S(═O)₂—R′ group, where R′ is as defined herein.

A “sulfonate” group refers to an —S(═O)₂—O—R′ group, where R′ is as defined herein.

A “sulfate” group refers to an —O-S(═O)₂—O—R′ group, where R′ is as defined as herein.

A “sulfonamide” or “sulfonamido” group encompasses both S-sulfonamido and N-sulfonamido groups, as defined herein.

An “S-sulfonamido” group refers to a —S(═O)₂—NR′R″ group, with each of R′ and R″ as defined herein.

An “N-sulfonamido” group refers to an R′S(═O)₂—NR″ group, where each of R′ and R″ is as defined herein.

An “O-carbamyl” group refers to an —OC(═O)—NR′R″ group, where each of R′ and R″ is as defined herein.

An “N-carbamyl” group refers to an R′OC(═O)—NR″— group, where each of R′ and R″ is as defined herein.

A “carbamyl” or “carbamate” group encompasses O-carbamyl and N-carbamyl groups.

An “O-thiocarbamyl” group refers to an —OC(═S)—NR′R″ group, where each of R′ and R″ is as defined herein.

An “N-thiocarbamyl” group refers to an R′OC(═S)NR″— group, where each of R′ and R″ is as defined herein.

A “thiocarbamyl” or “thiocarbamate” group encompasses O-thiocarbamyl and N-thiocarbamyl groups.

A “C-amido” group refers to a —C(═O)—NR′R″ group, where each of R′ and R″ is as defined herein.

An “N-amido” group refers to an R′C(═O)—NR″— group, where each of R′ and R″ is as defined herein.

A “urea” group refers to an —N(R′)—C(═O)—NR″R′″ group, where each of R′, R″ and R″ is as defined herein.

The term “thiourea” describes a —N(R′)—C(=S)—NR″R′″ group, where each of R′, R″ and R″ is as defined herein.

A “nitro” group refers to an —NO₂ group.

A “cyano” group refers to a —C═N group.

The term “phosphonyl” or “phosphonate” describes a —P(═O)(OR′)(OR″) group, with R′ and R″ as defined hereinabove.

The term “phosphate” describes an —O—P(═O)(OR′)(OR″) group, with each of R′ and R″ as defined hereinabove.

The term “phosphinyl” describes a —PR′R″ group, with each of R′ and R″ as defined hereinabove.

The term “hydrazine” describes a —NR′—NR″R′″ group, with R′, R″, and R″' as defined herein.

As used herein, the term “hydrazide” describes a —C(═O)—NR′—NR″R″′ group, where R′, R″ and R′″ are as defined herein.

As used herein, the term “thiohydrazide” describes a —C(=S)—NR′—NR″R″′ group, where R′, R″ and R′″ are as defined herein.

A “guanidinyl” group refers to an —RaNC(═NRd)—NRbRc group, where each of Ra, Rb, Rc and Rd can be as defined herein for R′ and R″.

A “guanyl” or “guanine” group refers to an RaRbNC(═NRd)— group, where Ra, Rb and Rd are as defined herein.

It is expected that during the life of a patent maturing from this application many relevant peptoids and uses thereof will be developed and the scope of the terms “peptoid” and “corresponding peptoid” are intended to include all such new technologies a priori.

As used herein the term “about” refers to ±10% The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Materials and Methods

Materials:

Rink amide resin was obtained from Novabiochem.

(S)-1-phenylethylamine (spe) and benzylamine (pm), piperazine (pz), and homopiperazine (hpz), were obtained from Acros Organics (Israel).

Bromoacetic acid, chloroacetic acid and N,N′-diisopropylcarbodiimide (DIC) were obtained from Sigma Aldrich.

Other reagents and solvents were obtained from commercial sources and used without additional purification.

Preparation of Peptoid Oligomers:

Peptoids were synthesized manually on a Rink amide resin using a submonomer approach such as described by Zuckermann et al. [J. Am. Chem. Soc. 1992, 114:10646-10647]. All peptoid oligomers were synthesized at room temperature. Typically, 100 mg of resin was swollen in dichloromethane (DCM) for 40 minutes before initiating oligomer synthesis. Multiple washing steps using dimethylformamide (DMF) were performed between each step described below. De-protection of resin was performed by addition of 20% piperidine solution (1.5 ml in DMF) and the reaction was allowed to shake at room temperature for 20 minutes. Following the reaction, piperidine was washed from the resin using DMF (10 ml per gram resin) (3×1 minute). Bromoacetylation was completed by adding 20 equivalents of bromoacetic acid (1.2 M in DMF, 8.5 ml per gram resin) and 24 equivalents of diisopropylcarbodiimide (2 ml per gram resin); this reaction was allowed to shake at room temperature for 20 minutes. Following the reaction, the bromoacetylation reagents were washed from the resin using DMF (10 ml per gram resin) (3×1 min) and 20 equivalents of submonomer amine (1.0 M in DMF, 10 ml per gram resin) were added. The amine displacement reaction was allowed to shake at room temperature for 20 minutes and was followed by multiple washing steps (DMF, 10 ml per gram resin) (3×1 min).

This two-step addition cycle was modified as follows:

To introduce piperazine scaffolds, initially, chloroacylation was completed by adding 20 equivalents chloroacetic acid (1.2 M in DMF, 8.5 ml per gram resin) and 24 equivalents of diisopropylcarbodiimide (2 ml per gram resin); this reaction was allowed to agitate at room temperature for 35 minutes. Following the reaction, the chloroacylation reagents were washed from the resin using DMF (10 ml per gram resin) (3×1 min) and 15 equivalents of piperazine (105 mg, 1.23 M in DMF, 10 ml per gram resin) was added. The amine displacement reaction was allowed to shake at room temperature for 45 minutes and was followed by multiple washing steps (DMF, 10 ml per gram resin) (3×1 min). Bromoacylations or chloroacylation and amine displacement steps were repeated until the desired peptoids were obtained. To cleave the peptoid oligomers from solid support for analysis, approximately 5 mg of resin was treated with 95% TFA in water (40 ml per gram resin) for 10 minutes. The cleavage cocktail was evaporated under nitrogen gas and the peptoid oligomers were re-suspended in 0.5 ml HPLC solvent (1:1 HPLC grade acetonitrile: HPLC grade water). To cleave the peptoid oligomers from solid support for preparative HPLC, the beads were treated with 5 ml of 95% TFA in water for 30 minutes. The cleavage cocktail was evaporated under low pressure, re-suspended in 2 ml HPLC solvent and lyophilized overnight.

Preparation of Cyclic Peptoid (P4):

100 mg of chloropropyl side chain of peptoid, 55 mg (6 equivalents) of KtBuO (potassium tert-butoxide, 2 ml dimethylformamide (DMF)+tetrahydrofuran (THF) were transferred into a microwave reactor. The reactor was subjected to microwave irradiation (Dynamic mode) for 60 minutes, 100 watt power and 60° C. Completion of the reaction was monitored by analytical HPLC. Upon completion of the reaction, the resin was washed with 2% HCl (2 ml), DMF and dichloromethane (DCM). To cleave the peptoid oligomers from solid support for analysis, approximately 5 mg of resin was treated with 95% trifluoroacetic acid (TFA) in water (40 ml per gram resin) for 10 minutes. The cleavage cocktail was evaporated under nitrogen gas and the peptoid oligomers were re-suspended in 0.5 ml HPLC solvent (1:1 HPLC grade acetonitrile: HPLC grade water). To cleave the peptoid oligomers from solid support for preparative HPLC, the beads were treated with 5 ml of 95% TFA in water for 30 minutes. The cleavage cocktail was evaporated under low pressure, re-suspended in 2 ml HPLC solvent and lyophilized overnight.

Preparation of β-alanine-based Peptoid β-peptoid) Oligomers:

100 mg of resin was swollen in dichloromethane (DCM) for 40 minutes before initiating oligomer synthesis. De-protection of resin was performed by addition of 20% piperidine solution (1.5 ml in dimethylformamide (DMF)) and the reaction was allowed to shake at room temperature for 20 minutes. Following the reaction, piperidine was washed from the resin using DMF (10 ml per gram resin) (3×1 minute). A solution of acryloyl chloride (6 equivalents) and N,N-diisopropylethylamine (12 equivalents) in DMF (5 ml) were added to a Rink amide resin. After shaking for 3 hours, the resin was drained and subsequently washed with DMF (3×3 ml), methanol (3×3 ml), and DCM (3×3 ml). A solution of submonomer amine (6 equivalents) in methanol (5 ml) was then added to the resin, which was shaken at 50° C. for 18 hours, and then washed as described above. To introduce piperazine, initially, chloroacylation was completed by adding 20 equivalents chloroacetic acid (1.2 M in DMF, 8.5 ml per gram resin) and 24 equivalents of diisopropylcarbodiimide (2 ml per gram resin); this reaction was allowed to agitate at room temperature for 35 minutes. Following the reaction, the chloroacylation reagents were washed from the resin using DMF (10 ml per gram resin) (3×1 minutes) and 15 equivalents of piperazine was added (105 mg, 1.23 M in DMF, 10 ml per gram resin, after each substitution reaction with piperazine washed with hot DMF, 10 ml per gram resin (3×1 minute)). After amine displacement, steps were repeated until the desired peptoids were obtained, and the crude product was then cleaved from solid support for analysis. To cleave the peptoid oligomers from solid support for analysis, approximately 5 mg of resin was treated with 50% trifluoroacetic acid (TFA) in DCM (40 ml per gram resin) for 10 minutes. The cleavage cocktail was evaporated under nitrogen gas and the peptoid oligomers were re-suspended in 0.5 ml HPLC solvent (1:1 HPLC grade acetonitrile: HPLC grade water). To cleave the peptoid oligomers from solid support for preparative HPLC the beads were treated using 50% TFA in DCM for 2×30 minutes. The cleavage cocktail was evaporated under low pressure, re-suspended in 2 ml HPLC solvent and lyophilized overnight.

Preparation of N-carbamyl-containing Peptoid (Azapeptoid) Oligomers:

For the synthesis of azapeptoids containing N-carbamyl-containing side chains, Knorr Rink amide (RAM) resin was used. Knorr RAM resin (100 mg) in 2 ml dimethylformamide (DMF) was swollen at room temperature for 1 hour. Fmoc protecting group was then removed with 20% piperidine in DMF (2 ml) for 30 minutes and the resin was subsequently washed with DMF (3×3 ml). After Fmoc group removal, 2-bromoacetic acid (1 ml, 2 M in DMF) and diisopropylcarbodiimide (1 ml, 3.4 M in DMF) were added. The reaction vessel containing the resin was stirred for about 10 minutes at 37° C. and subsequently washed with DMF (3×3 ml). A solution of amine/carbazate (2 ml, 2 M in N-methylpyrrolidone) was then added and stirred at 37° C. for 1 hour. To introduce piperazine, initially, chloroacylation was completed by adding 20 equivalents chloroacetic acid (1.2 M in DMF, 8.5 ml per gram resin) and 24 equivalents of diisopropylcarbodiimide (2 ml per gram resin); this reaction was allowed to agitate at room temperature for 35 minutes. Following the reaction, the chloroacylation reagents were washed from the resin using DMF (10 ml per gram resin) (3×1 min) and 15 equivalents of piperazine was added (105 mg, 1.23 M in DMF, 10 ml per gram resin, after each substitution reaction with piperazine washed with hot DMF, 10 ml per gram resin (3×1 minute)). The bromoacylation and S_{N}2 displacement reactions were repeated to obtain the desired chain length. After amine displacement, steps were repeated until the desired peptoids were obtained, and the crude product was then cleaved from solid support for analysis. To cleave the peptoid oligomers from solid support for analysis, approximately 5 mg of resin was treated with 96% trifluoroacetic acid (TFA), 2% triisopropylsilane (TIS) and 2% water (40 ml per gram resin) for 10 minutes. The cleavage cocktail was evaporated under nitrogen gas and the peptoid oligomers were re-suspended in 0.5 ml HPLC solvent (1:1 HPLC grade acetonitrile: HPLC grade water). The resin was treated with the cleavage cocktail of 96% TFA, 2% TIS and 2% water for 1 hour at room temperature. The cleavage cocktail solution was then dried by flushing argon, and cold ether was added to precipitate out the compounds, which were then subjected to reverse phase HPLC to confirm the purity of the products.

General Procedure for Solution Phase Synthesis of Short Compounds:

An amine (13 mmol) and 2.092 ml (15 mmol) TEA (triethylamine) were added to 30 ml of dry dichloromethane (DCM) in a flame-dried, three-necked flask with a magnetic bar. The flask was purged with N₂, and cooled to −78° C. in a dry ice/acetone bath. Bromoacetyl bromide (14 mmol, 1.219 ml) in DCM (10 ml) was added drop-wise via a syringe to the stirring solution under a gentle stream of N₂. After completing the addition, the reaction mixture was stirred at −78° C. for 30 minutes, then removed from the bath and stirred for 6 hours at room temperature. Thereafter, the mixture was transferred to a separatory funnel, washed with 10% (w/v) aqueous citric acid and saturated bicarbonate solution. The compound was dried with anhydrous sodium carbonate and solvent was evaporated and crude compound was used as such for next step. The obtained corresponding amido bromide (2 mmol) and K₂CO₃ (10 mmol) were dissolved in 50 ml acetonitrile and 1 mmol of piperazine was added; and the mixture was refluxed for 6 hours. Solvent was evaporated and washed with a mixture of water and dichloromethane (DCM) and brine, and then dried with anhydrous sodium carbonate. After evaporating the DCM, the compound was washed with cold diethyl ether three times to obtain pure compounds. This technique does not require column chromatography for purification.

Crystallization:

Short peptoid-like compounds (100 mg) were dissolved in 10 ml diethyl ether/methanol which was allowed to evaporate slowly at room temperature. A colorless single crystal was obtained in diethyl ether solvent was found to be suitable for the X-ray.

General Method for Water Solubility Test:

1 mg peptoid was placed in an Eppendorf container and water was added gradually (e.g., 5 pi per addition) until a clear solution was obtained. The solubility test was repeated three times and average values are presented. The limit of the test was typically 2.0×10² mg/liter (i.e., 1 mg per 5 ml).

Circular Dichroism (CD):

Circular dichroism (CD) measurements were performed using a J-810-150S circular dichroism spectropolarimeter (Jasco). Spectra were obtained in a 1 mm path-length fused quartz cell. Prior to taking measurements, 5 mM stock oligomer solution was constituted in 100% water. The appropriate dilutions were made immediately before the measurements. Eight scans were taken for each sample at a scan rate of 1 nm/second at 1 nm increments. Data are expressed in terms of per-residue molar ellipticity (degrees*cm²/dmol) calculated per mole of amide groups present.

UV-visible Spectrometry:

UV-visible spectrometry measurements were performed using a Cary™ 60 UV-Vis spectrophotometer (Agilent) with Czerny-Turner monochromator. In a typical UV-visible experiment, 10 μl of a peptoid solution (5 mM) was diluted in 3 ml H₂O (to obtain a 17 μM concentration) and the UV-visible spectrum was measured. Afterwards 1 equivalent of Cu²⁺ (10 μl, 5 mM) or solution containing mixtures of metal ions (1 equivalent Cu²⁺, Co²⁺, Zn²⁺, Mn²⁺, Ni²⁺ and Fe³⁺, 5 mM, 10 μl) was added and the spectrum was measured again.

Mass Spectrometry:

Mass spectrometry (MS) was performed on an LCT Premier™ mass spectrometer (Water) and an expression™ mass spectrometer (Advion) under electrospray ionization (ESI), direct probe ACN:H₂O (70:30). Reactions were monitored and analyzed by a 3800 gas chromatograph (Varian) using a CPSil-8 column. All peptoids were subjected to ESI-MS characterization. Calculated molar mass and molar mass as determined by mass spectrometry for synthesized peptoids are presented in Table 1 below.

TABLE 1
Peptoid sequences and confirmation of sequences by mass spectrometry

| | | Molar | Molar | Crude |
|---|---|---|---|---|
| Peptoid | | mass | mass | purity |
| (sequences are towards N-terminus) | | (calculated) | (measured) | (%) |
| P1 | (Nspe)₆ | 984.26 | 984.80 | 99 |
| SP1 | (Npm)₆-Nea | 1000.19 | 1000.32 | 83 |
| SP2 | (Npm)₆-Neh | 1001.18 | 1002.04 | 79 |
| SP3 | (Nspe)₆-Naa | 1211.47 | 1211.56 | 80 |
| SP4 | (Nspe)₆-Nabs | 1099.32 | 1099.96 | 81 |
| P2 | (Nspe)₆-Npz | 1110.41 | 1110.8 | 85 |
| P3 | Npz-(Nspe)₆ | 1110.41 | 1110.8 | 81 |
| P-linear | Npl-(Nspe)₆-Npz | 1243.94 | 1243.45 | 70 |
| P4 | Npl-(Nspe)₆-Npz (cyclic) | 1207.50 | 1207.12 | 40 |
| P5 | (Nspe)₆-(Npz)₂ | 1236.57 | 1236.96 | 82 |
| P6 | (Nspe)₆-(Npz)₃ | 1224.64 | 1124.69 | 85 |
| P7 | (Nspe)₆-Nhpz | 1224.64 | 1124.69 | 85 |
| P8 | (Nspe)₆-(Nhpz)₂ | 1264.63 | 1264.69 | 82 |
| P9 | (Ns1npe)₄ | 861.07 | 861.89 | 99 |
| P11 | (Npm)₂-Nsch-Ncpe-Nsch-Nfpm | 1020.77 | 1020.55 | 90 |
| P12 | (Ns1npe)₄-Npz | 988.22 | 988.83 | 82 |
| P13 | Npz-(Ns1npe)₄ | 988.22 | 988.52 | 80 |
| P14 | (Ns1npe)₄-Nhpz | 1002.25 | 1003.00 | 81 |
| P15 | (Nsch)₅-Npz | 979.43 | 979.20 | 75 |
| P16 | (Nsch)₅-Nhpz | 993.45 | 993.74 | 76 |
| P17 | Npz-(Npm)₂-Nsch-Ncpe-Nsch-Nfpm | 1146.89 | 1145.49 | 70 |
| P18 | (Ns1npe)₄-(Npz)₂ | 1114.38 | 1114.67 | 78 |
| P19 | (Nsch)₅-(Npz)₂ | 1105.58 | 1105.86 | 72 |
| P20 | (Npz)₂-(Npm)₂-Nsch-Ncpe-Nsch-Nfpm | 1273.02 | 1272.33 | 47 |
| P21 | (Ns1npe)₄-(Npz)₃ | 1240.54 | 1240.26 | 72 |
| P22 | (Nsch)₅-(Npz)₃ | 1231.74 | 1231.87 | 67 |
| P23 | (Npz)₃-(Npm)₂-Nsch-Ncpe-Nsch-Nfpm | 1273.02 | 1272.33 | 47 |
| P33 | Nspe-Ntrp-(Nspe)₂-Nhq-Nspe-Npz | 1334.56 | 1334.34 | 80 |
| P34 | Npz-Nspe-Ntrp-(Nspe)₂-Nhq-Nspe | 1334.56 | 1334.20 | 72 |

Naa = glycine-N-acetic acid
Nabs = N-[p-sulfobenzyl]glycine;
Ncpe = N-[2-(p-chlorophenyl)ethyl]glycine
Nea = N-[2-aminoethyl]glycine
Neh = N-[2-hydroxyethyl]glycine
Nfpm = N-methyl-N-[1-(o-fluorophenyl)ethyl]glycine
Nhpz = N-homopiperazinyl acetic acid
Npl = N-[3-chloropropyl]glycine
Npz = N-piperazinyl acetic acid
Npm = N-[phenylmethyl]glycine
Nsch = N-[(S)-1-cyclohexylethyl]glycine
Ns1npe = N-[(S)-1-naphthylethyl]glycine
Nspe = N-[(S)-1-phenylethyl]glycine

High-performance Liquid Chromatography (HPLC):

Peptoid oligomers (P1 to P34) were analyzed by reversed-phase HPLC (analytical C18 column, 5 μm, 100 Å, 2.0×50 mm) on a UV-2075 instrument (Jasco). A linear gradient of 5% to 95% acetonitrile (ACN) in water (0.1% trifluoroacetic acid) over 10 minutes was used at a flow rate of 0.7 ml/minute (solvent A: 0.1% trifluoroacetic acid in HPLC grade water, solvent B: (0.1% trifluoroacetic acid in HPLC grade acetonitrile). Preparative HPLC was performed using a C18 column (Phenomenex) (15 μm, 100 Å 21.20×100 mm) on a UV-2075 instrument (Jasco). Peaks were eluted with a linear gradient of 5% to 95% ACN in water (0.1% trifluoroacetic acid) over 50 minutes at a flow rate of 5 ml/minute.

The peptoids were further purified to over 95% by reverse phase HPLC and lyophilized overnight.

NMR Spectroscopy:

¹H NMR spectra were recorded on a 400 MHz NMR instrument (Bruker) in CD₃OD (deuterated methanol). Coupling constants are given in Hz. The following abbreviations are used to indicate the multiplicity: s, singlet; d, doublet; m, multiplet; bs, broad signal.

### Example 1

### Effect of Piperazine Groups on Peptoid Water Solubility

Peptoid hexamers were prepared incorporating only a single hydrophilic group, namely, a carboxylic acid, an alcohol, a primary amine or a sulfonic acid at the N-terminus. The structures of these peptoid hexamers are presented in FIG. 1.

As shown in FIG. 2, peptoid hexamers comprising a carboxylic acid, alcohol, primary amine or sulfonic acid group at the N-terminus were not soluble in water.

The possibility of incorporating polar group(s) within the backbone of the peptoid (at either terminal), and the effect of such modifications on water solubility was then studied. Piperazine, which is biocompatible, was chosen as a polar group.

As depicted in FIG. 3, the two secondary amine units of piperazine, which are equally reactive towards bromoacetic acid, can both react simultaneously to connect two peptoid oligomers, thereby forming a “peptoid dimer” (PD), or alternatively, piperazine can react with a single peptide, resulting in a “peptoid monomer” (PM).

As further shown in FIG. 3, a bromoacetylated short peptoid trimer containing methyl benzyl synthons (P) reacted with (10 equivalents) piperazine at the N-terminus (using typical “submonomer” conditions) afforded only 60% of the desired PM and 40% of PD, as determined by HPLC analysis. In contrast, using chloroacetic acid instead of bromoacetic acid improved the product ratio to 80% PM and 20% PD, and increasing the amount of piperazine from 10 equivalents to 15 equivalents further improved the product ratio to 85% PM and 15% PD.

These results are consistent with reports that replacing bromoacetic acid with chloroacetic acid in peptoid synthesis improves the selectivity and efficiency of the reaction [Burkoth et al., J. Am. Chem. Soc. 2003, 125:8841-8845] because the relative reaction rate of a chloride leaving group is slower than that of a bromide [Bateman et al., J. Chem. Soc. 1940, 979-1011].

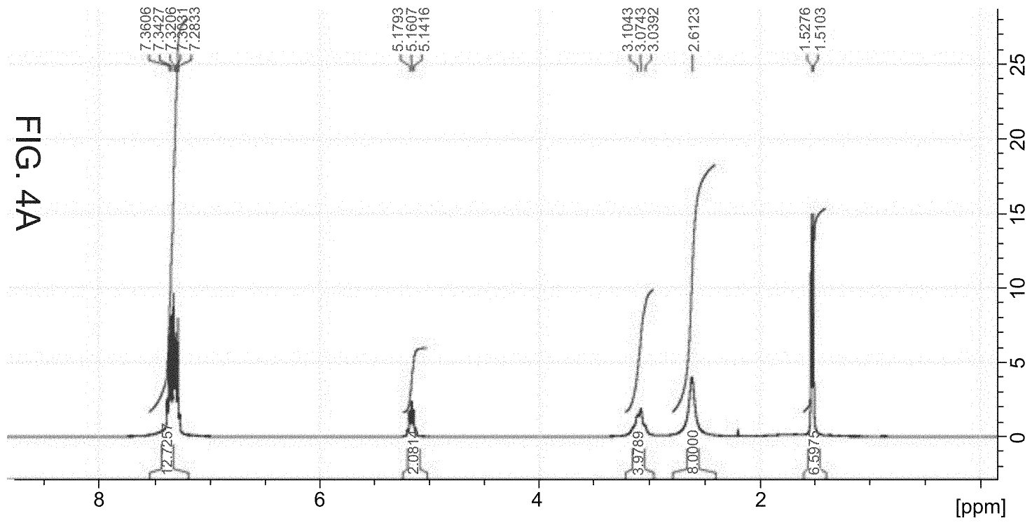

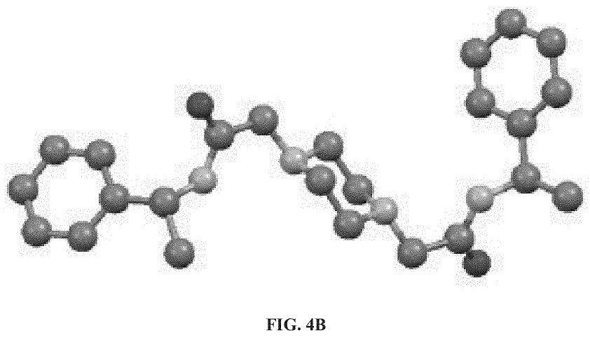

Attempts to crystalize PM or PD were not successful. A short peptoid “dimer” PD′ bearing two (S)-(-)-1-phenylethylamine (Nspe) groups were therefore synthesized, using a solution phase method as described hereinabove. The structure of PD' was confirmed by ¹H-NMR spectroscopy (as shown in FIG. 4A), according to procedures described hereinabove, and the compound was crystallized, and its crystal structure was determined by X-ray analysis.

As shown in FIG. 4B, the piperazine of PD′ is in the chair conformation, and the two Nspe groups are facing one up and one down relative to the plain of the piperazine ring.

A hydrophobic helical peptoid oligomer (P1) was synthesized as an example for a water-insoluble peptoid (water solubility<2.0×10² mg/liter), and peptoids P2 and P3 were designed and prepared in order to evaluate the effect of piperazine on water-solubility of P1 when placed either at the N-terminus or C-terminus of P1, and peptoid P4 was designed and prepared in order to test the effect of piperazine incorporated within a cyclic hydrophobic peptoid. The structures of the peptoids P1-P4 are presented in FIG. 5. The linear peptoids P1, P2 and P3, as well as P-linear (an intermediate in the preparation of the cyclic P4), which incorporate piperazine, Nspe and chloropropylamine (Np1, only in P-linear) as synthons, were synthesized on solid support employing the “submonomer” protocol. Peptoid P4 was prepared by a microwave-assisted cyclization of the peptoid P-linear on solid support, according to procedures described in Kaniraj & Maayan [Org. Lett. 2015, 17:2110-2113]. All the peptoids were analyzed and purified (to over 95% purity) by HPLC, and their sequences were confirmed by electrospray ionization mass spectrometry (ESI-MS).

Water solubility tests were carried out by a gradual addition of 5 μl distilled water to 1 mg peptoid until a clear solution was obtained. This assay was repeated three times and results were averaged.

As shown in FIG. 6, modification of the water-insoluble P1 peptoid with piperazine greatly enhanced the water solubility thereof (in contrast to peptoids modified with other polar groups, as shown in FIG. 2), with the modified peptoids being completely water-soluble at a relatively high concentration of about 0.9 M upon the insertion of even one piperazine at either its N-terminus (P2) or C-terminus P3). Furthermore, the cyclic peptoid P4 was also water-soluble, albeit in a lower concentration of about 0.7 M, perhaps due to its lack of a secondary amine available for hydrogen bonding with water.

The peptoids P2-P4 exhibited water-solubility over a considerable time period, and no precipitation was detected even after several months in which their solutions were allowed to stand undisturbed at room temperature.

As shown in FIG. 7, P2 exhibited high water solubility at acidic and near neutral pH ((1.66±0.16)×10³ mg/liter at pH 4.5 and (1.28±0.14)×10³ mg/liter at pH 7.5), and lower water solubility at basic pH (<2.0×10² mg/liter at pH 10.5), which is consistent with the water solubility of piperazine per se at different pH values.

The above results show that piperazine is extremely effective at enhancing water solubility of peptoids, especially in comparison with various other polar groups, when incorporated at the N-terminus or C-terminus or within the backbone of a peptoid.

### Example 2

### Factors Associated with Enhancement of Peptoid Water Solubility by Piperazine

Two main factors may contribute to the capability of piperazine to solubilize modified P1 in water:

(1) the presence of two sites (the two nitrogen atoms) available for hydrogen bonding with water, as compared to only one such site that is present in the other polar groups tested; and

(2) an ability of piperazine, when placed at the N-terminus of P1, to form intramolecular hydrogen bonding between the two nitrogen atoms, which may result in less intermolecular hydrogen bonding between P2 oligomers, thereby facilitating formation of hydrogen bonds with water molecules.

In order to explore the influence of the first factor on the water solubility of piperazine-modified peptoids, two more peptoids (P5 and P6) were synthesized, having two and three piperazine units, respectively, at their N-terminus, as depicted in FIG. 8. These peptoids were purified, their sequences were confirmed by ESI-MS, and their water solubility was evaluated according to procedures described hereinabove.

The water solubility of P5 ((5.80±0.11)×10⁴ mg/liter) was over 50 times higher than that of P2 ((1.00±0.15)×10³ mg/liter), and the water solubility of P6 ((8.86±0.06)×10⁴ mg/liter) was even higher than that of P5.

Furthermore, as shown in FIG. 9, at pH 10.5, the water solubilities of P5 and P6 were 30 times and 106 times higher, respectively, than that of P2.

As the additional piperazine groups in P5 and P6 do not add secondary amines to the sequences, these results indicate that the increase in water solubility arises from the addition of nitrogen atoms, capable for hydrogen bonding with water. These results support the hypothesis that the number of nitrogen atoms is an important factor in enabling piperazine groups to solubilize a peptoid in water.

In order to assess the role of the second factor mentioned hereinabove (intramolecular hydrogen bonding between nitrogen atoms), piperazine (a six-membered ring) was replaced by homopiperazine (a seven-membered ring analogous to piperazine, a.k.a. diazepane). As depicted in FIG. 10, intramolecular hydrogen bonding in piperazine is possible only when the piperazine group is in the energetically unfavorable boat conformation (rather than the more favorable chair conformation), the hydrogen bond forming a five-membered ring; whereas homopiperazine is most stable in the twist chair conformation, which enables intramolecular hydrogen bonding while forming a six membered ring. It was therefore hypothesized that replacing piperazine with homopiperazine would result in greater water solubility if intramolecular hydrogen bonding plays an important role.

The homopiperazine-containing peptoids P7 and P8 (as depicted in FIG. 11) were synthesized, purified and analyzed, and their solubility in water was evaluated according to procedures described hereinabove.

The water solubility of P7 ((4.96±0.07)×10⁴ mg/liter) was about 50 times higher than that of P2 ((1.00±0.15)×10³ mg/liter)—comparable to the water solubility of P5 ((5.80±0.11)×10⁴ mg/liter)—and the water solubility of P8 ((5.10±0.12)×10⁵ mg/liter) was even higher than that of P7 and even that of P6 ((8.86±0.06)×10⁴ mg/liter).

These results indicate that homopiperazine is considerably more effective than piperazine at enhancing water solubility, with one homopiperazine (as in P7) at the N-terminus of a peptoid having a similar effect as two piperazines (as in P5), and two homopiperazines (as in P8) having a stronger effect than three piperazines (as in P6).

Taken together, the above results indicate that the number of nitrogen atoms and intramolecular hydrogen bonding between hydrogen atoms are both important factors in the water solubility of a peptoid modified with piperazine or homopiperazine.

In order to confirm that modification as described herein can be utilized to solubilize a variety of hydrophobic peptoids in water, the peptoids P9, P10 and P12 (depicted in FIG. 12) were modified by addition of piperazine or homopiperazine at the N-terminus. P9, a tetramer bearing four (S)-(-)-1-naphthylethylamine (N1snp) groups, and P10, a pentamer having five (S)-(-)-1-cyclohexylethylamine (Nsch) groups, were both previously reported, and their helical structures, which also resemble the polyproline I (PPI) type helix, was determined by X-ray crystallography [Stringer et al., J. Am. Chem. Soc. 2011, 133:15559-15567; Wu & Kirshenbaum, J. Am. Chem. Soc. 2015, 137:6312-6319]. P11 was design in accordance with the sequence of a biologically active peptoid recently described by Trader et al. [J. Am. Chem. Soc. 2015, 137:6312-6319].

As shown in Table 2 below, none of P9-P11 was water-soluble, and peptoids P12-P17 formed by incorporation of one piperazine or homopiperazine unit into P9-P11 also did not exhibit water solubility. As further shown therein, water solubility was observed only upon insertion of two piperazine units into P9-P11 (peptoids P18-P20), with even greater water solubility being observed upon incorporation of three piperazine units (peptoids P21-P23).

TABLE 2
Effect of piperazine and homopiperazine on peptoid water solubility

| | | Water |
|---|---|---|
| | | solubility |
| Peptoid | | (mg/liter) |
| P9 | (Ns1npe)₄ | <2.0 × 10² |
| P10 | (Nsch)₅ | <2.0 × 10² |
| P11 | (Npm)₂-Nsch-Ncpe-Nsch-Nfpm | <2.0 × 10² |
| P12 | (Ns1npe)₄-piperazine | <2.0 × 10² |
| | (P9-piperazine) | |
| P13 | piperazine-(Ns1npe)₄ | <2.0 × 10² |
| | (piperazine-P9) | |
| P14 | (Ns1npe)₄-homopiperazine | <2.0 × 10² |
| | (P9-homopiperazine) | |
| P15 | (Nsch)₅-piperazine | <2.0 × 10² |
| | (P10-piperazine) | |
| P16 | (Nsch)₅-homopiperazine | <2.0 × 10² |
| | (P10-homopiperazine) | |
| P17 | piperazine-(Npm)₂-Nsch-Ncpe-Nsch-Nfpm | <2.0 × 10² |
| | (piperazine-P11) | |
| P18 | (Ns1npe)₄-(piperazine)₂ | (1.33 ± 0.14) × 10⁴ |
| | (P9-(piperazine)₂) | |
| P19 | (Nsch)₅-(piperazine)₂ | (7.52 ± 0.11) × 10³ |
| | (P10-(piperazine)₂) | |
| P20 | (piperazine)₂-(Npm)₂-Nsch- | (8.44 ± 0.10) × 10⁴ |
| | Ncpe-Nsch-Nfpm | |
| | ((piperazine)₂-P11) | |
| P21 | (Ns1npe)₄-(piperazine)₃ | (1.76 ± 0.07) × 10⁵ |
| | (P9-(piperazine)₃) | |
| P22 | (Nsch)₅-(piperazine)₃ | (7.35 ± 0.09) × 10⁴ |
| | (P10-(piperazine)₃) | |
| P23 | (piperazine)₃-(Npm)₂-Nsch- | (1.35 ± 0.07) × 10⁵ |
| | Ncpe-Nsch-Nfpm | |
| | ((piperazine)₃-P11) | |

Ncpe = N-[2-(p-chlorophenyl)ethyl]glycine
Nfpm = N-methyl-N-[1-(o-fluorophenyl)ethyl]glycine
Npm = N-[phenylmethyl]glycine
Nsch = N-[(S)-1-cyclohexylethyl]glycine
Ns1npe = N-[(S)-1-naphthylethyl]glycine

The above results demonstrate the generality of the use of one or more piperazine units and similar units to enhance water solubility of hydrophobic peptoids.

In order to assess whether the dissolved peptoids form aggregates, fluorescence spectra were determined for P18 and P21 (which comprise Ns1npe) dissolved in water. Aggregation of peptoids with Ns1npe groups has been reported to be associated with an excimer band at 392 nm [Fuller et al., Org. Lett. 2013, 15:5118-5121]. In addition, CD spectra of P21 were determined at various concentrations ranging from 40 to 180 μM in water.

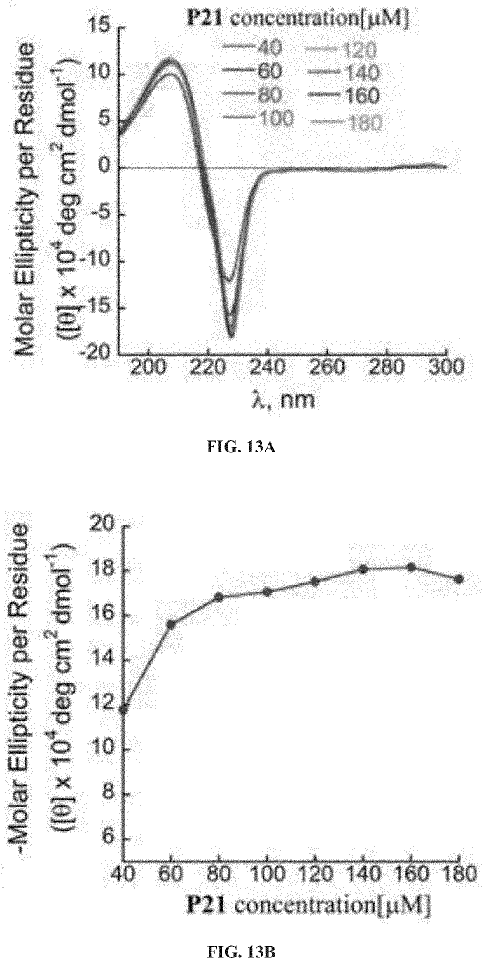

As shown in FIGS. 13A and 13B, some increase was observed in the intensity of the band near 228 nm in the CD spectrum of P21 at increased concentrations, suggesting the possibility of some concentration-dependent aggregation of P21 in water.

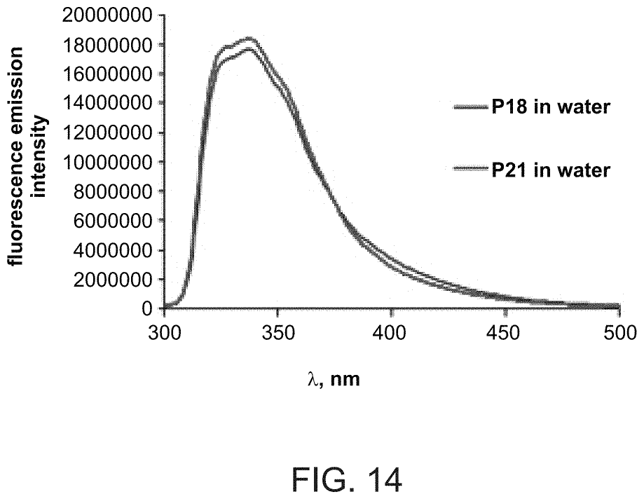

However, as shown in FIG. 14, no excimer band was observed near 392 nm for either P18 or P21.

Similarly, CD spectra of P2, P5 and P8 (which comprise Nspe rather than Ns1npe as in P18 and P21) were determined in order to assess whether the dissolved peptoids form aggregates in water.

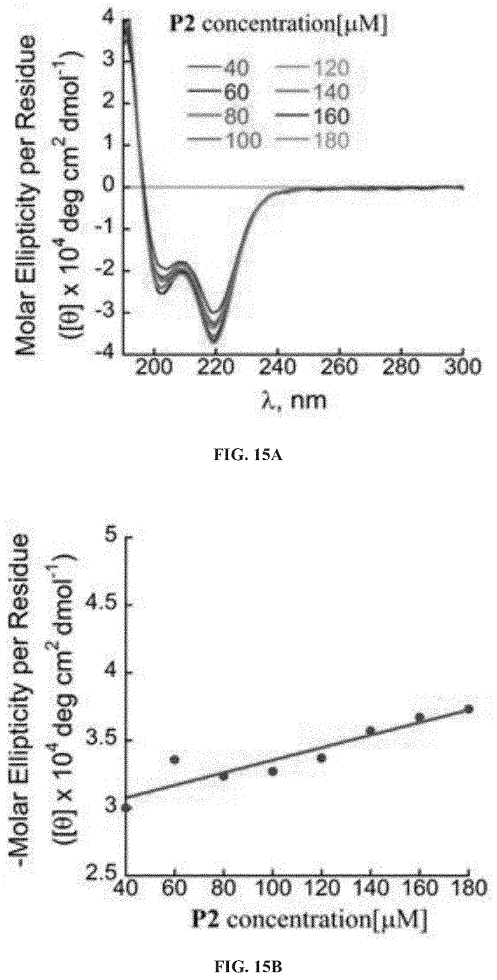

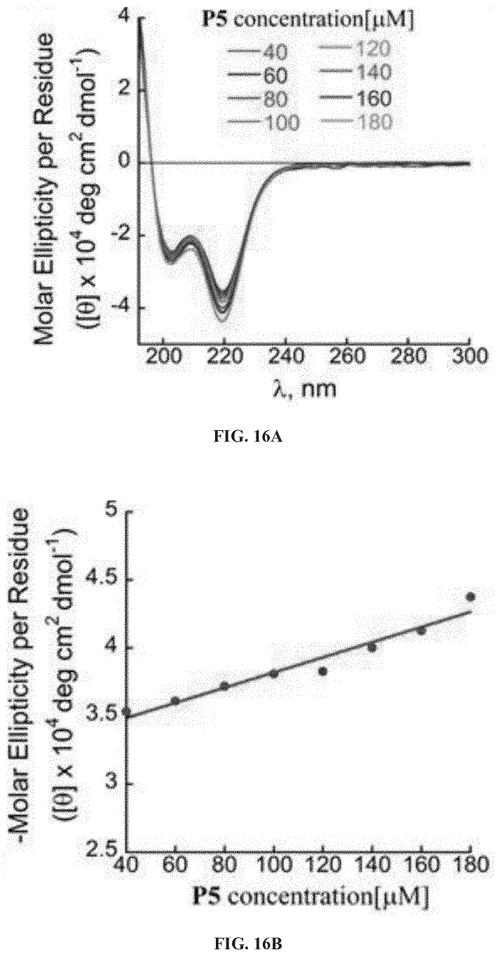

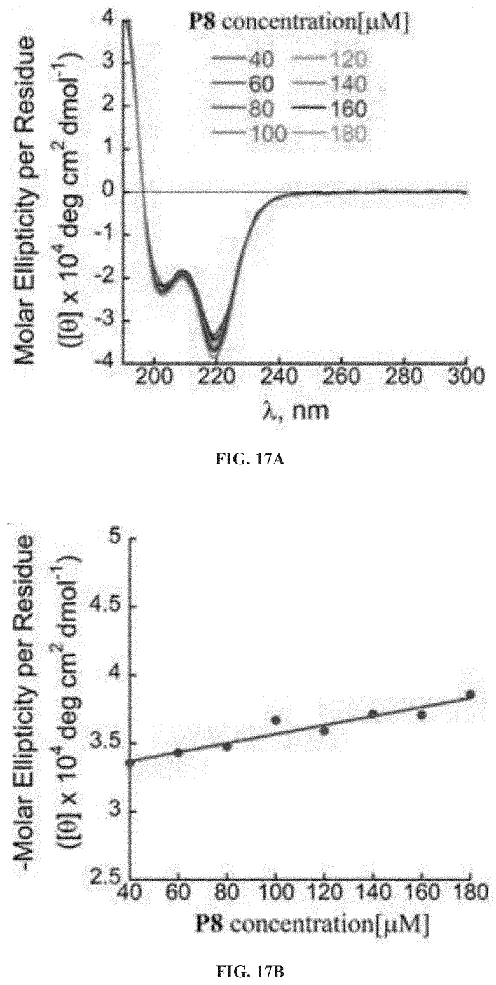

As shown in FIGS. 15A-17B, almost no concentration-dependent increase was observed in the minimum band near 219 nm for P2 (FIGS. 15A and 15B), P5 (FIGS. 16A and 16B) or P8 (FIGS. 17A and 17B), suggesting that no aggregation of these peptoids occurred in water.

Taken together, these results indicate that only limited aggregation of the peptoids, if any, occurs in water, and that most of the hydrophobic interactions are intramolecular rather than intermolecular.

### Example 3

### Effect of Piperazine Groups on Peptoid Structure

The secondary structures of the P1 and P9 peptoids, and modified variations thereof, were studied in water and in other solvents, using circular dichroism (CD).

As shown in FIG. 18, the Nspe peptoid P1 exhibits a characteristic CD spectrum with bands near 190 and 202 nm.

The 202 nm band has been associated with the trans-amide bond conformation, with the cis-amide bond conformation being associated with a 218 nm band [Wu et al., J. Am. Chem. Soc. 2001, 123:2958-2963]. P1 adopts a right-handed helix that resembles the PPI type helix.

As further shown in FIG. 18, both peptoids P2 and P3 exhibit CD spectra in acetonitrile with an intense band at 218 nm (red and brown lines, respectively), indicating that addition of piperazine at the N-terminus of P1 increases its conformational order, leading to a solution in which helices with only cis-amide bonds are the major population. In addition, the CD spectra of P2 and P3 in water were comparable to these obtained in acetonitrile, indicating that the helical structure associated with the sequence of P1 is maintained also in water.

As shown in FIG. 19, the CD spectrum of P2 in water does not change significantly upon increasing the temperature from 0° C. to 90° C., indicating that the helical structure is stable in water and does not deform as a function of temperature.

As shown in FIG. 7, the CD spectrum of P2 at pH 4.5 (in Tris buffer) exhibited double minima at about 202 and 218 nm, characteristic of the helical structure. As further shown therein, such double minima were also obtained in the CD spectrum at pH 10.5, albeit in a much lower intensity.

In comparison, as shown in FIG. 9, the CD spectrum of P6, which is highly soluble at pH 10.5, exhibited band intensity similar to that observed for P2 at acidic and near neutral pH (as shown in FIG. 7).

Taken together, these results indicate that the low intensity of CD bands for P2 at pH 10.5 can be attributed to the lower solubility of P2 in basic solution.

As shown in FIG. 20, the CD spectra of peptoids P7 and P8 exhibit an intense band at 218 nm in both acetonitrile and water, similarly to the spectra of P2 and P3.

These results suggest an increase in the conformational order of P1 upon the addition of one or two homopiperazine groups at its N-terminus, while maintaining the helical structure in water.

As shown in FIG. 21, the CD spectra of P18 and P21 in acetonitrile resemble the CD spectrum of P9, with a broad maximum band near 205 nm, a reduced minimum band near 220 nm and an intense minimum band near 230 nm. These results indicate that the peptoid structure did not change upon incorporation piperazine groups into P9.

However, as further shown in FIG. 21, the CD spectra of P18 and P21 in water changed (relative to spectra in acetonitrile) such that the band near 220 nm disappeared and the band near 230 nm became twice as intense.

The abovementioned spectra P18 and P21 in water are actually similar to the ones reported for longer N1snp homo oligomers (in acetonitrile), and the increase in the intensity of this band was associated with higher conformational order and the formation of a robust helical structure [Stringer et al., J. Am. Chem. Soc. 2011, 133:15559-15567].

These results indicate that the addition of piperazine to a helical peptoid can remarkably result in a water-soluble peptoid with an extreme helix stability.

### Example 4

### Modification of Additional Peptoids with Piperazine

In order further assess the effect of piperazine groups on water solubility of various peptoids, 1, 2 or 3 piperazine groups were added to the N-terminus of N-(1-phenylethyl)-O-alanine hexamer, a representative β-alanine-based peptoid (prepared according to procedures described hereinabove); or to the N-terminus of hexamer of alternating N-(1-phenylethyl)glycine and N-(ethoxycarbonylamino)glycine residues, a representative peptoid comprising N-carbamyl side chains (prepared according to procedures described hereinabove).

As shown in Table 3 below, peptoids formed by incorporation of two or three piperazine units into an N-(1-phenylethyl)-β-alanine hexamer exhibit water solubility comparable to that of analogous piperazine-modified N-(1-phenylethyl)glycine hexamers (e.g., P5 and P6).

As further shown therein, peptoids formed by incorporation of one, two or three piperazine units into a hexamer of alternating N-(1-phenylethyl)glycine and N-(ethoxycarbonylamino)glycine residues exhibit a high degree of water solubility.

These results further demonstrate the generality of the use of one or more piperazine units and similar units to enhance water solubility of hydrophobic peptoids, including peptoids comprising N-substituted β-alanine residues and peptoids comprising carbamyl side chains (e.g., as a substituent of a glycine nitrogen atom).

TABLE 3
Effect of piperazine on peptoid water solubility

| | | Water solubility |
|---|---|---|
| Peptoid | | (mg/liter) |
| P24 | 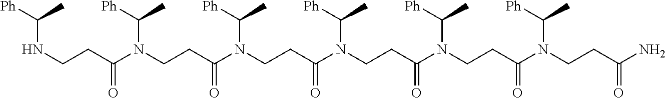 [SMILES: C[C@H](c1ccccc1)N(CCC(N)=O)C(=O)CCN(C(=O)CCN(C(=O)CCN(C(=O)CCN(C(=O)CCN[C@H](C)c1ccccc1)[C@H](C)c1ccccc1)[C@H](C)c1ccccc1)[C@H](C)c1ccccc1)[C@H](C)c1ccccc1] | <2.0 × 10² |
| P25 | 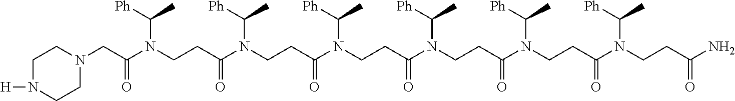 [SMILES: C[C@H](c1ccccc1)N(CCC(N)=O)C(=O)CCN(C(=O)CCN(C(=O)CCN(C(=O)CCN(C(=O)CCN(C(=O)CN1CCNCC1)[C@H](C)c1ccccc1)[C@H](C)c1ccccc1)[C@H](C)c1ccccc1)[C@H](C)c1ccccc1)[C@H](C)c1ccccc1] | <2.0 × 10² |
| P26 | 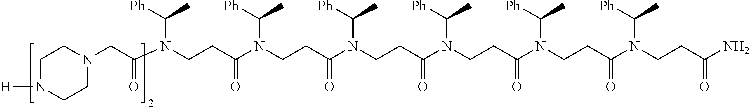 | (1.03 ± 0.09) × 10⁵ |
| P27 | 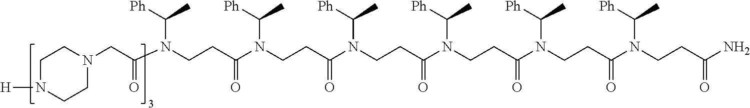 | (1.96 ± 0.11) × 10⁵ |
| P28 | 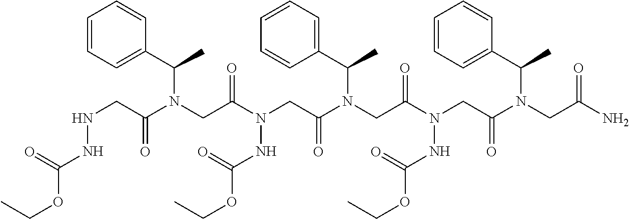 [SMILES: CCOC(=O)NNCC(=O)N(CC(=O)N(CC(=O)N(CC(=O)N(CC(=O)N(CC(N)=O)[C@H](C)c1ccccc1)NC(=O)OCC)[C@H](C)c1ccccc1)NC(=O)OCC)[C@H](C)c1ccccc1] | <2.0 × 10² |
| P29 | 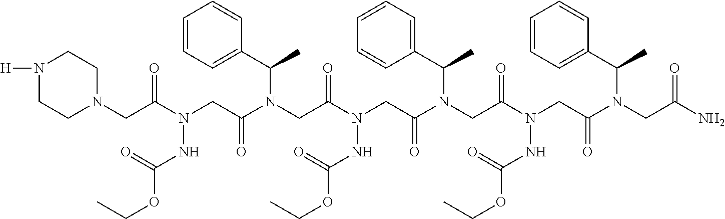 [SMILES: CCOC(=O)NN(CC(=O)N(CC(N)=O)[C@H](C)c1ccccc1)C(=O)CN(C(=O)CN(NC(=O)OCC)C(=O)CN(C(=O)CN(NC(=O)OCC)C(=O)CN1CCNCC1)[C@H](C)c1ccccc1)[C@H](C)c1ccccc1] | (5.06 ± 0.13) × 10⁴ |
| P30 | 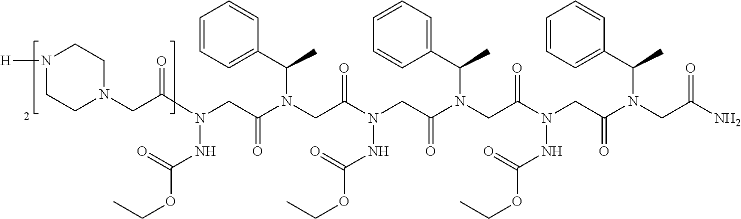 | (1.77 ± 0.09) × 10⁵ |
| P31 | 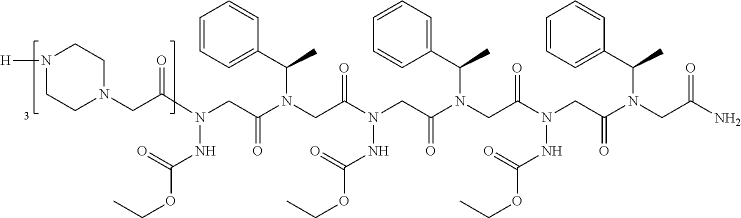 | (1.68 ± 0.06) × 10⁶ |

β-Peptoids containing various side chains generally exhibit cis-trans isomerization equilibria in solution similar to their peptoid analogs [Laursen et al., Nat. Commun. 2015, 6:7013; Laursen et al., Acc. Chem. Res. 2015, 48:2696-2704; Norgren et al., Org. Lett. 2006, 8:4533-4536; Olsen et al., Amino Acids 2008, 34:465-471].

As shown in FIG. 22, the CD spectra in acetonitrile for the β-peptoids P24-P27 show a characteristic double minima near 200 and 220 nm, albeit with the latter being less intense than in the case of α-peptoids, in accordance with literature reports on phenyl β-peptoids [Laursen et al., Nat. Commun. 2015, 6:7013; Laursen et al., Acc. Chem. Res. 2015, 48:2696-2704].

As further shown in FIG. 22, the CD spectra of the water soluble β-peptoids P26 and P27 (with two or three piperazine residues, respectively) exhibit a much more intense negative absorbance band near 220 nm. These results suggest that, as in the case of P9, intramolecular hydrophobic interactions in water give rise to considerably higher conformational order of the helical structures.

In contrast to α-peptoids and β-peptoids, in which the tertiary amide bond exhibits a strong preference for the cis geometry, azapeptoids have been reported to exist almost entirely in the trans conformation, as determined by solution ¹H-NMR measurements and X-ray crystallography [Sarma & Kodadek, ACS Comb. Sci. 2012, 14:558-564; Sarma et al., Chem. Commun. 2011, 47:10590-10592].

As shown in FIG. 23, the CD spectra in acetonitrile for the azapeptoids P28-P31 exhibited one intense minimum at 195 nm. These results suggest absence of an azapeptoid secondary structure such as a helical structure.

As further shown in FIG. 23, the CD spectra of the water soluble azapeptoids P29-P31 (with one, two or three piperazine residues, respectively) exhibited an additional absorbance band near 220 nm. These results indicate an increase in the population of conformers with cis-amide bond(s) that results in the initial formation of a helical structure.

The above results indicate that simple incorporation of one or two piperazine units can enhance structure formation in water of a wide variety of peptoids, including initiating structure formation of unstructured peptoids.

### Example 5

### Functional Activity of Piperazine-modified Peptoid Chelator in Water

The effect on functional activity in water of a peptoid by addition of one or two piperazine or homopiperazine group(s) at the N- or C-terminus of the peptoid sequence was assessed.

P32, a recently reported selective peptoid chelator for Cu²⁺ [Baskin & Maayan, Chem. Sci. 2016, 7:2809-2820], was modified by addition of a single piperazine group at either its N-terminus or C-terminus, to form the modified peptoids P33 and P34, respectively. The structures of P32, P33 and P34 are presented in FIG. 24.

Although P32 is water-insoluble (water solubility<2.0×10² mg/liter) and thus relatively useless for practical applications, the water solubility of P33 was (2.44±0.08)×10⁵ mg/liter, and the water solubility of P34 was (8.30±0.15)×10³ mg/liter.

As shown in FIG. 25, the secondary structure of the piperazine-modified peptoid P34 was maintained in water, indicating that the solubilization of peptoids in water does not destroy secondary structure.

In order to evaluate the selectivity of P33 and P34 towards Cu²⁺, 1 equivalent of each peptoid in water was added to 1 equivalent of a mixture solution containing the metal ions Cu²⁺, Co²⁺, Zn²⁺, Fe²⁺, Mn²⁺ and Ni²⁺, and UV-visible absorption spectra were recorded and compared to UV-visible absorption of Cu²⁺-complexes of the peptoids. It has been previously shown that comparing UV-visible absorption spectra is a highly reliable tool to assess the selectivity of the peptoid chelators.

As shown in FIG. 26, the UV-visible spectrum of P33 contacted with a mixture of metal ions was different from the spectrum of the P33 Cu²⁺-complex, suggesting that P33 does not selectively bind to Cu²⁺.

In contrast, as shown in FIG. 27, the UV-visible spectrum of P34 contacted with a mixture of metal ions was identical to the spectrum of the P34 Cu²⁺-complex, indicating that P34 selectively binds to Cu²⁺.

These different results suggest an ability of piperazine, when incorporated at the N-terminus (e.g., as opposed to the C-terminus) of a peptoid, to participate in the binding of some metal ions and disable the unique coordination geometry of the Cu²⁺ complex that allows its selective binding.

Taken together, the above results indicate that piperazine modification can solubilize peptoids in water as well as preserve in water selective functional activities of peptoids, such as ion binding.

### Example 6

### Modification of Peptoids with Piperazine Analogs

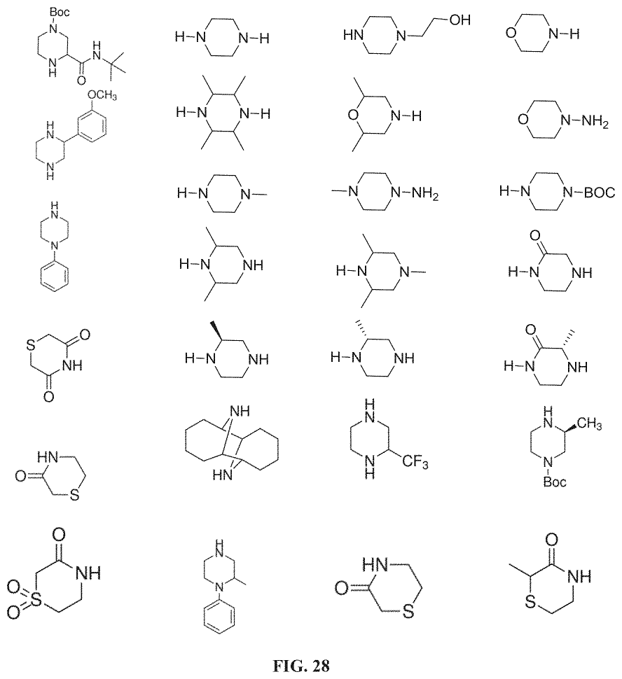

Modified peptoids are prepared according to procedures described in any of Examples 1-5 described hereinabove, except that a compound (piperazine analog) depicted in FIG. 28 is used instead of piperazine (or homopiperazine).

The effect of the piperazine analog on water solubility of the modified peptoid is optionally evaluated according to procedures described hereinabove (e.g., in Example 1 or 2). The structure and/or functional activity of the modified peptoid is optionally evaluated according to procedures described in Examples 3 and 5, respectively.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. (canceled)

2. A peptoid comprising a plurality of N-substituted glycine residues and/or N-substituted β-alanine residues, and at least one heteroalicyclic residue having a general formula: 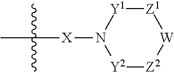
wherein:
W is selected from the group consisting of NR₁₅, O, S, S(═O), S(═O)2, and a nitrogen atom attached by a covalent bond to another portion of the peptoid;
X is selected from the group consisting of —C(═O)—CR₁R₂— and —NR_{16—}, or X is absent;
Y¹ is CR₃R₄ or C═O;
Y² is CR₅R₆ or C═O;
Z¹ is CR₇R₈ or C═O;
Z² is CR₉R₁₀ or CR₁₁R₁₂—CR₁₃R₁₄;
R₁-R₁₄ are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, phosphate, phosphonyl, phosphinyl, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, hydrazine, and amino; and
R₁₅ and R₁₆ are each independently selected from the group consisting of hydrogen, alkyl, alkenyl, alkynyl, cycloalkyl, aryl, heteroaryl, heteroalicyclic, carbonyl, thiocarbonyl, C-amido, and C-carboxy,
or alternatively, any two of R₁-R₁₆ together form a 5-, 6- or 7-membered alicyclic or heteroalicyclic ring.

3. (canceled)

4. The peptoid of claim 2, wherein R₁-R₁₆ are each independently hydrogen or methyl.

5. The peptoid of claim 2, wherein at least one of said at least one heteroalicyclic residue is directly attached via an amide bond to a backbone nitrogen atom or a backbone carbonyl of at least one of said N-substituted glycine residues and/or N-substituted β-alanine residues.

6. The peptoid of claim 2, wherein at least one heteroalicyclic residue is a terminal residue.

7-8. (canceled)

9. The peptoid of claim 2, wherein Z² is CR₁₁R₁₂—CR₁₃R₁₄.

10. (canceled)

11. The peptoid of claim 2, wherein Y¹ is CR₃R₄ and Y² is CR₅R₆.

12. (canceled)

13. The peptoid of claim 2, comprising at least two of said heteroalicyclic residue.

14. The peptoid of claim 13, wherein said at least two of said heteroalicyclic residue are attached to one another.

15. The peptoid of claim 2, comprising at least one of said heteroalicyclic residue per 750 Da of the total weight of the peptoid.

16-17. (canceled)

18. The peptoid of claim 2, comprising at least three of said N-substituted glycine residues and/or N-substituted β-alanine residues.

19. The peptoid of claim 2, wherein R₃-R₁₄ are each independently selected from the group consisting of hydrogen, alkyl, aryl, and C-amido, or alternatively, R₃ and R₅, and/or R₇ and R₉, together form a 5-, 6- or 7-membered heteroalicyclic ring.

20-21. (canceled)

22. The peptoid of claim 2, having a water-solubility of at least 200 mg/liter.

23. The peptoid of claim 2, wherein a water-solubility of a corresponding peptoid lacking said at least one heteroalicyclic residue is less than 100 mg/liter.

24. The peptoid of claim 2, wherein a water-solubility of the peptoid is at least 150% of a water-solubility of a corresponding peptoid lacking said at least one heteroalicyclic residue.

25. The peptoid of claim 2, being characterized by a helical structure in aqueous solution.

26. The peptoid of claim 2, wherein a corresponding peptoid lacking said at least one heteroalicyclic residue is characterized by a helical structure in acetonitrile.

27. The peptoid of claim 2, wherein at least a portion of said N-substituted glycine residues and/or N-substituted β-alanine residues comprise a side chain which comprises a moiety selected from the group consisting of aryl, heteroaryl, cycloalkyl and heteroalicyclic.

28-31. (canceled)

32. A composition comprising the peptoid of claim 2 and an aqueous liquid, said peptoid being soluble in said aqueous liquid.

33. The composition of claim 32, wherein a concentration of said peptoid in the aqueous liquid is at least 10 mg/liter.

34. The composition of claim 32, wherein a pH of the aqueous liquid solution is in a range of from 5 to 9.
